(19) United States
(12) Patent Application Publication
(10) Pub. No.: US 2025/0304705 A1
(43) Pub. Date: Oct. 2, 2025

(54) BIOTIN ORTHOGONAL STREPTAVIDIN SYSTEM

(71) Applicant: UNIVERSITY OF UTAH RESEARCH FOUNDATION, Salt Lake City, UT (US)

(72) Inventors: Michael S. KAY, Salt Lake City, UT (US); Steven R. E. Draper, Provo, UT (US)

(21) Appl. No.: 18/863,298

(22) PCT Filed: May 15, 2023
(86) PCT No.: PCT/US2023/067028

§ 371 Date: Nov. 5, 2024

Related U.S. Application Data
(60) Provisional application No. 63/342,052, filed on May 13, 2022.

(51) Int. Cl. C07K 16/28; A61K 45/06; A61K 47/68; A61K 49/00; A61K 51/04; C07K 14/36

(52) U.S. Cl. CPC C07K 16/2878; A61K 45/06; A61K 47/6898; A61K 49/00; A61K 51/0453; C07K 14/36; C07K 2317/55; C07K 2317/622; C07K 2319/00

(57) ABSTRACT

The present disclosure relates to an orthogonal system comprising a first bi-specific polypeptide that comprises D-streptavidin or a variant thereof covalently linked to an antibody or antibody fragment and a second bi-specific polypeptide that comprises L-biotin covalently linked to a therapeutic or diagnostic agent. The disclosed systems can be useful in, for example, treating a disease or a condition (e.g., cancer, non-Hodgkin lymphoma, multiple sclerosis, Crohn's disease, rheumatoid arthritis, asthma, macular degeneration, psoriasis, Hodgkin lymphoma, paroxysmal nocturnal hemoglobinuria, X-linked hypophosphatemia). Also described are peptides and polypeptides useful in preparing the disclosed bi-specific polypeptides and methods of making same. This abstract is intended as a scanning tool for purposes of searching in the particular art and is not intended to be limiting of the present invention.

### CROSS-REFERENCE TO RELATED APPLICATIONS

This Application claims the benefit of U.S. Application No. 63/342,052, filed on May 13, 2022, the contents of which are incorporated herein by reference in their entirety.

### REFERENCE TO SEQUENCE LISTING

The Sequence Listing submitted May 12, 2023 as a xml file named “21101.0438P1_Updated.xml,” created on May 3, 2023, and having a size of 20,480 bytes is hereby incorporated by reference pursuant to 37 C.F.R. § 1.52(e)(5).

### BACKGROUND

Streptavidin (SA) evolved in Streptomyces avidinii (S. avidinii) to impede the growth of competing bacteria by sequestering biotin.^{24,25} SA forms a complex network of hydrogen bonds and other noncovalent interactions with biotin,^{26,27} forming the strongest known small molecule-protein noncovalent interaction in nature with a K_{D} in the femtomolar range (4.8×10⁻⁴ M).¹ This extraordinary binding affinity has led to multiple applications of SA in biomedical research, including in immunoassays,² affinity chromatography,²⁸ proximity labeling,^{4,5} and phage display.²⁹ Additionally, SA/biotin-based diagnostic tests⁶ are widely used (e.g., for heart disease and thyroid conditions).

Though SA/biotin enjoys great success in a variety of biotechnology applications, several challenges limit its utility, especially in therapeutic contexts. For example, biotin supplementation can lead to interference in diagnostics that use SA and biotin.³⁰⁻³³ Likewise, proximity labeling is a powerful recent technology that employs a non-specific biotin ligase to tag molecules in close proximity to ID binding partners.^{4,5} However, biotinylation by endogenous biotin ligases leads to background noise that limits identification of low-abundance targets.⁴ Most importantly for therapeutic applications, streptavidin is a highly immunogenic foreign protein.^{3,20, 34-36}

There has been much interest in using SA for pretargeted immunotherapy (PTI).⁷⁻⁹ The concept of PTI evolved from targeted immunotherapy (TI),¹⁹ where a radioactive payload is attached to an antibody that targets a tumor specific antigen. The antibody-conjugate binds and focuses the irradiation on the tumor cell, but unbound antibodies can stay in circulation for several days,^{9,10,20,35} all the while irradiating and damaging healthy tissues.^{9,14} To overcome this problem, PTI methods involve conjugating high-affinity binding partners to the antibody and to the drug. The antibody is administered prior to the drug, allowing it to bind to the tumor cell without irradiating healthy tissue. After unbound antibody is cleared from circulation, the drug is injected. The drug associates with its binding partner to concentrate at the tumor site. Since the drug is a small molecule, the excess clears from circulation rapidly, sparing healthy tissue.

PTI has been approached in four ways: (1) with bispecific antibodies,^{34,36,37} where one arm of the antibody binds to the tumor cell and the other arm binds to the drug; (2) with SA and biotin (SA PTI),^{34,36,37} where streptavidin is conjugated to the antibody and the drug is biotinylated; (3) with oligonucleotides^{36,37} with one strand attached to the antibody and the complimentary strand attached to the drug; and (4) using high-speed click reactions,^{20,34,36,37} such as inverse electron demand Diels-Alder chemistry (IEDDAC).³⁵ When compared head-to-head with other methods, SA PTI has been the least effective. SA is immunogenic and antibodies clear it from circulation rapidly.^{3,20,34-36} Bispecific antibodies have higher therapeutic efficacy and lower immunogenicity.³ IECCAC has fast kinetics (3×10⁴ M⁻¹s⁻¹)³⁵ and the benefit of forming a covalent bond. However, each of these methods have serious drawbacks. Bispecific antibodies are challenging to produce^{3,24,25} and have weak affinity for the drug they bind.³⁶ Oligonucleotides³⁵ and IEDDAC methods have low in vivo stability.³⁵ SA-biotin binding is up to 1000 times faster than IECCAC.^{35,38} Additionally, though IECCAC forms a covalent bond, SA's high affinity and low off-rate make it a nearly irreversible interaction.^{1,39} In summary, SA PTI has great potential, but its effectiveness is diminished by endogenous biotin interference^{3,16,20,34,36} and extreme immunogenicity.^{3,20,34-36}

D-Proteins offer an elegant solution to the problems with SA PTI. A D-protein represents the mirror-image, or enantiomer, of its naturally occurring L-counterpart. According to the law of mirror-image symmetry, a D-protein interaction with its mirror-image ligand must have the same binding affinity as its naturally occurring L-counterpart. Additionally, D-proteins cannot be proteolytically degraded²¹ for MHC presentation to the immune system,²² significantly increasing their half-lives in circulation. Thus, a system based on L-biotin and D-SA has the potential to dramatically advance the PTI field. Unfortunately, synthetic methods to access D-SA that are scaleable and reproduceable have remained elusive. Thus, there remains a need for methods of synthesizing D-SA and variants thereof, conjugates comprising these D-proteins, and methods of using the conjugates in, inter alia, PTI. These needs and others are met by the following disclosure.

### SUMMARY

In accordance with the purpose(s) of the invention, as embodied and broadly described herein, the invention, in one aspect, relates to an orthogonal system comprising a first bi-specific polypeptide that comprises D-SA or a variant thereof covalently linked to an antibody or antibody fragment and a conjugate that comprises L-biotin covalently linked to a therapeutic or diagnostic agent. The disclosed systems can be useful in, for example, treating a disease or a condition (e.g., cancer, non-Hodgkin lymphoma, multiple sclerosis, Crohn's disease, rheumatoid arthritis, asthma, macular degeneration, psoriasis, Hodgkin lymphoma, paroxysmal nocturnal hemoglobinuria, X-linked hypophosphatemia). Also described are peptides and polypeptides useful in preparing the disclosed bi-specific polypeptides and methods of making same.

Thus, in one aspect, disclosed are peptides comprising an amino acid sequence of at least 90% identity to the amino acid sequence AEAGITGTWYNQLGSTFIVTAGADGALTGTYES (SEQ ID NO:1), wherein each amino acid in the amino acid sequence is a D-amino acid or wherein each amino acid in the amino acid sequence is a L-amino acid.

In one aspect, disclosed are peptides comprising an amino acid sequence of at least 90% identity to the amino acid sequence AVGNAESRYVLTGRYDSAPATDGSGTALGWTVAWKNNYRNAHS (SEQ ID NO:3), wherein each amino acid in the amino acid sequence is a D-amino acid or wherein each amino acid in the amino acid sequence is a L-amino acid.

In one aspect, disclosed are peptides comprising an amino acid sequence of at least 90% identity to the amino acid sequence ATTWSGQYVGGAEARINTQWLLTSGTTEANAWKSTLVGHDTFTKVKPSAAS (SEQ ID NO:5), wherein each amino acid in the amino acid sequence is a D-amino acid or wherein each amino acid in the amino acid sequence is a L-amino acid.

In one aspect, disclosed are peptides comprising an amino acid sequence of at least 90% identity to the amino acid sequence XHHAEAGITGTWYNQLGSTFIVTAGADGALTGTYES-N₂H₄(SEQ ID NO:6), wherein each amino acid in the amino acid sequence is a D-amino acid or wherein each amino acid in the amino acid sequence is a L-amino acid, and wherein X_{HH} is a linker.

In one aspect, disclosed are peptides comprising an amino acid sequence of at least 90% identity to the amino acid sequence KKKKKKX_{HH}AEAGiTGTWYNQLGSTFIVTAGADGALTGTYES-N₂H₄(SEQ ID NO:7), wherein each amino acid in the amino acid sequence is a D-amino acid or wherein each amino acid in the amino acid sequence is a L-amino acid, and wherein KKKKKK and X_{HH}together comprise a solubilizing residue.

In one aspect, disclosed are polypeptides comprising an amino acid sequence that has from 90% to 99% identity to the amino acid sequence AEAGITGTWYNQLGSTFIVTAGADGALTGTYES AVGNAESRYVLTGRYDSAPATDGSGTALGWTVAWKNNYRNAHS ATTWSGQYVGGAEARINTQWLLTSGTTEANAWKSTLVGHDTFTKVKPSAAS (SEQ ID NO:8), wherein each amino acid in the amino acid sequence is a D-amino acid.

In one aspect, disclosed are polypeptides comprising an amino acid sequence of at least 90% identity to the amino acid sequence KKKKKKX_{HH}AEAGITGTWYNQLGSTFIVTAGADGALTGTYES AVGNAESRYVLTGRYDSAPATDGSGTALGWTVAWKNNYRNAHS-N₂H₄(SEQ ID NO:9), wherein each amino acid in the amino acid sequence is a D-amino acid or wherein each amino acid in the amino acid sequence is a L-amino acid, and wherein KKKKKK and X_{HH} together comprise a solubilizing residue.

In one aspect, disclosed are polypeptides comprising an amino acid sequence of at least 90% identity to the amino acid sequence KKKKKKX_{HH}AEAGITGTWYNQLGSTFIVTAGADGALTGTYES AVGNAESRYVLTGRYDSAPATDGSGTALGWTVAWKNNYRNAHS ATTWSGQYVGGAEARINTQWLLTSGTTEANAWKSTLVGHDTFTKVKPSAAS (SEQ ID NO:10), wherein each amino acid in the amino acid sequence is a D-amino acid or wherein each amino acid in the amino acid sequence is a L-amino acid, and wherein KKKKKK and X_{HH} together comprise a solubilizing residue.

In one aspect, disclosed are methods of making L-SA, D-SA, or a variant thereof, the method: (a) providing a first peptide having an amino acid sequence that has at least 90% identity to the amino acid sequence AEAGITGTWYNQLGSTFIVTAGADGALTGTYES-N₂H₄(SEQ ID NO:2), wherein each amino acid in the amino acid sequence is a D-amino acid or wherein each amino acid in the amino acid sequence is a L-amino acid; (b) providing a second peptide having an amino acid sequence that has at least 90% identity to the amino acid sequence AVGNAESRYVLTGRYDSAPATDGSGTALGWTVAWKNNYRNAHS-N₂H₄(SEQ ID NO:4), wherein each amino acid in the amino acid sequence is a D-amino acid or wherein each amino acid in the amino acid sequence is a L-amino acid; (c) providing a third peptide having an amino acid sequence that has at least 90% identity to the amino acid sequence ATTWSGQYVGGAEARINTQWLLTSGTTEANAWKSTLVGHDTFTKVKPSAAS (SEQ ID NO:5), wherein each amino acid in the amino acid sequence is a D-amino acid or wherein each amino acid in the amino acid sequence is a L-amino acid; and (d) ligating the first, second, and third peptides.

In one aspect, disclosed are methods of making L-SA, D-SA, or a variant thereof, the method comprising: (a) coupling a first peptide having an amino acid sequence that has at least 90% identity to the amino acid sequence AEAGITGTWYNQLGSTFIVTAGADGALTGTYES-N₂H₄(SEQ ID NO:2), wherein each amino acid in the amino acid sequence is a D-amino acid or wherein each amino acid in the amino acid sequence is a L-amino acid, and a linker; (b) functionalizing the linker with a positively charged amino acid residue, thereby providing a solubilizing residue; (c) ligating the first peptide to a second peptide having an amino acid sequence that has at least 90% identity to the amino acid sequence AVGNAESRYVLTGRYDSAPATDGSGTALGWTVAWKNNYRNAHS-N₂H₄(SEQ ID NO:4), wherein each amino acid in the amino acid sequence is a D-amino acid or wherein each amino acid in the amino acid sequence is a L-amino acid; (d) ligating the second peptide to a third peptide having an amino acid sequence that has at least 90% identity to the amino acid sequence ATTWSGQYVGGAEARINTQWLLTSGTTEANAWKSTLVGHDTFTKVKPSAAS (SEQ ID NO:5), wherein each amino acid in the amino acid sequence is a D-amino acid or wherein each amino acid in the amino acid sequence is a L-amino acid; and (e) cleaving the solubilized residue from the polypeptide.

In one aspect, disclosed are methods of making a polypeptide comprising an amino acid sequence that has at least 90% identity to the amino acid sequence KKKKKKX_{HH}AEAGITGTWYNQLGSTFIVTAGADGALTGTYES AVGNAESRYVLTGRYDSAPATDGSGTALGWTVAWKNNYRNAHS ATTWSGQYVGGAEARINTQWLLTSGTTEANAWKSTLVGHDTFTKVKPSAAS (SEQ ID NO:10), wherein each amino acid in the amino acid sequence is a D-amino acid or wherein each amino acid in the amino acid sequence is a L-amino acid, and wherein KKKKKK and X_{HH} together comprise a solubilizing residue, the method comprising: (a) providing a first polypeptide having an amino acid sequence that has at least 90% identity to the amino acid sequence KKKKKKX_{HH}AEAGITGTWYNQLGSTFIVTAGADGALTGTYES AVGNAESRYVLTGRYDSAPATDGSGTALGWTVAWKNNYRNAHS-N₂H₄(SEQ ID NO:9), wherein each amino acid in the amino acid sequence is a D-amino acid or wherein each amino acid in the amino acid sequence is a L-amino acid; (b) providing a third peptide having an amino acid sequence that has at least 90% identity to the amino acid sequence ATTWSGQYVGGAEARINTQWLLTSGTTEANAWKSTLVGHDTFTKVKPSAAS (SEQ ID NO:5), wherein each amino acid in the amino acid sequence is a D-amino acid or wherein each amino acid in the amino acid sequence is a L-amino acid; and (c) ligating the first polypeptide to the third peptide.

In one aspect, disclosed are methods of making L-SA, D-SA, or a variant thereof, the method comprising: (a) providing a polypeptide having an amino acid sequence that has at least 90% identity to the amino acid sequence KKKKKKX_{HH}AEAGITGTWYNQLGSTFIVTAGADGALTGTYES AVGNAESRYVLTGRYDSAPATDGSGTALGWTVAWKNNYRNAHS ATTWSGQYVGGAEARINTQWLLTSGTTEANAWKSTLVGHDTFTKVKPSAAS (SEQ ID NO:10), wherein each amino acid in the amino acid sequence is a D-amino acid or wherein each amino acid in the amino acid sequence is a L-amino acid, and wherein KKKKKK and X_{HH} together comprise a solubilizing residue; and (b) cleaving a solubilized residue from the polypeptide.

In one aspect, disclosed are bi-specific polypeptides comprising a single chain antibody (scFv) or a F′ab fragment and a disclosed polypeptide.

In one aspect, disclosed are bi-specific polypeptides comprising an antibody or antibody fragment thereof covalently linked to D-SA or a variant thereof.

In one aspect, disclosed are conjugates comprising L-biotin covalently linked to a therapeutic agent or a diagnostic agent.

In one aspect, disclosed are pharmaceutical compositions comprising an effective amount of a disclosed bi-specific polypeptide and a pharmaceutically acceptable carrier.

In one aspect, disclosed are methods of treating a disease or a condition in a subject in need thereof, the method comprising: (a) administering to the subject an effective amount of a disclosed bi-specific polypeptide; and (b) subsequently administering to the subject an effective amount of a composition comprising L-biotin covalently linked to a therapeutic agent, wherein the D-SA or the variant thereof specifically binds L-biotin.

In one aspect, disclosed are kits comprising a disclosed bi-specific polypeptide, and one or more selected from: (a) a therapeutic agent; (b) L-biotin; (c) instructions for administering the bi-specific polypeptide in connection with treating a disease or a condition; and (d) instructions for treating the disease or the condition.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows a representative scheme depicticting four pre-targeting techniques (PTI): bispecific antibody PTI (A), SA PTI (B), oligonucleotide PTI (C), and click chemistry PTI (D).

FIG. 2A and FIG. 2B are representative plots showing ITC data. Specifically, FIG. 2A shows representative ITC data for the titration of L-SA with D-biotin. FIG. 2A shows representative ITC data for the titration of L-SA with L-biotin.

FIG. 3 shows a representative scheme illustrating the native chemical ligation (NCL) reaction: transthio-esterification, where the sulfur on cysteine performs a nucleophilic attack on carbonyl of the thioester, then S- to N-acyl shift to from a native peptide.

FIG. 4 shows a representative scheme depicting a Helping Hand (HH) molecule. The helping hand is added to a primary amine in the peptide and the functionalized with positively charged residues to increase solubility. The tag can be scarlessly removed after.

FIG. 5 shows a representative scheme illustrating the synthesis strategy for streptavidin. SA1, SA2, and SA3 sequences are shown. Underlined Ala will be formed by desulfurizing Cys after NCL.

FIG. 6A and FIG. 6B show representative plots illustrating representative spectral data of full-length synthetic L-SA. Specifically, FIG. 6A is a representative mass spectrum.

FIG. 6B is a representative RP-HPLC chromatogram.

FIG. 7A-F show representative plots for the LC-MS characterization of SA1, SA2, and SA3. Specifically, FIG. 7A is a representative RP-HPLC chromatogram corresponding to SAL. FIG. 7B is a representative mass spectrum corresponding to SAL. FIG. 7C is a representative RP-HPLC chromatogram corresponding to SA2. FIG. 7D is a representative mass spectrum corresponding to SA2. FIG. 7E is a representative RP-HPLC chromatogram corresponding to SA3. FIG. 7F is a representative mass spectrum corresponding to SA3.

FIG. 8A and FIG. 8B are representative plots for the LC-MS characterization of SA1 and SA2 ligation product. Specifically, FIG. 8A is a representative RP-HPLC chromatogram corresponding to the SA1 and SA2 ligation product. FIG. 8B is a representative mass spectrum corresponding to the SA1 and SA2 ligation product.

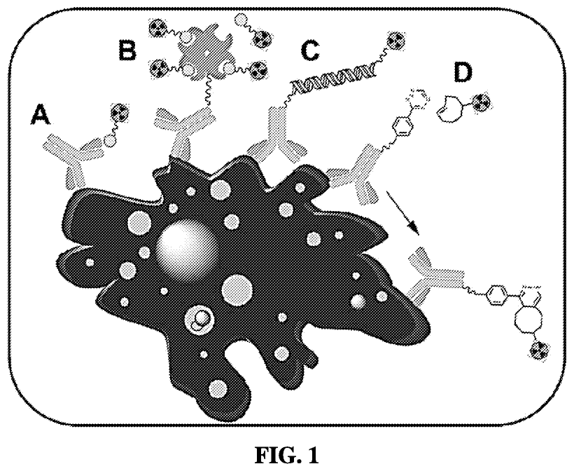

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present peptides, compositions, and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used in the specification and the appended claims, the singular forms “a,” “an,” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated f10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the fatty acids, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific fatty acid employed; the duration of the treatment; drugs used in combination or coincidental with the specific fatty acid employed and like factors well known in the medical arts. For example, it is well within the skill of the art to start doses of a fatty acid at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed fatty acid, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed fatty acid, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; anti-cancer and anti-neoplastic agents such as kinase inhibitors, poly ADP ribose polymerase (PARP) inhibitors and other DNA damage response modifiers, epigenetic agents such as bromodomain and extra-terminal (BET) inhibitors, histone deacetylase (HDAc) inhibitors, iron chelotors and other ribonucleotides reductase inhibitors, proteasome inhibitors and Nedd8-activating enzyme (NAE) inhibitors, mammalian target of rapamycin (mTOR) inhibitors, traditional cytotoxic agents such as paclitaxel, dox, irinotecan, and platinum compounds, immune checkpoint blockade agents such as cytotoxic T lymphocyte antigen-4 (CTLA-4) monoclonal antibody (mAB), programmed cell death protein 1 (PD-i)/programmed cell death-ligand 1 (PD-L1) mAB, cluster of differentiation 47 (CD47) mAB, toll-like receptor (TLR) agonists and other immune modifiers, cell therapeutics such as chimeric antigen receptor T-cell (CAR-T)/chimeric antigen receptor natural killer (CAR-NK) cells, and proteins such as interferons (IFNs), interleukins (ILs), and mAbs; anti-ALS agents such as entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

A As used herein the terms “amino acid” and “amino acid identity” refers to one of the 20 naturally occurring amino acids or any non-natural analogues that may be in any of the antibodies, variants, or fragments disclosed. Thus, “amino acid” as used herein means both naturally occurring and synthetic amino acids. For example, homophenylalanine, citrulline and norleucine are considered amino acids for the purposes of the invention. “Amino acid” also includes amino acid residues such as proline and hydroxyproline. The side chain may be in either the (R) or the (S) configuration. In some aspects, the amino acids are in the D- or L-configurations as further described herein. If non-naturally occurring side chains are used, non-amino acid substituents may be used, for example, to prevent or retard—degradation.

As used herein, the term “polypeptide” refers to a polymer composed of amino acid residues related to naturally occurring structural variants, and synthetic non-naturally occurring analogs thereof linked via peptide bonds or modified peptide bonds (i.e., peptide isosteres), related naturally occurring structural variants, and synthetic non-naturally occurring analogs thereof, glycosylated polypeptides, and all “mimetic” and “peptidomimetic” polypeptide forms. Synthetic polypeptides can be synthesized, for example, using an automated polypeptide synthesizer. The term can refer to an oligopeptide, peptide, polypeptide, or protein sequence, or to a fragment, portion, or subunit of any of these. The term “protein” typically refers to large polypeptides. The term “peptide” typically refers to short polypeptides. In various aspects, each amino acid residue in the polypeptide or polymer can be a D-amino acid such as, for example, in a D-protein (e.g., D-SA). Alternatively, in various further aspects, each amino acid residue in the polypeptide or polymer can be a D-amino acid such as, for example, in a D-protein (e.g., L-SA).

A “portion” of a polypeptide or protein means at least about three sequential amino acid residues of the polypeptide. It is understood that a portion of a polypeptide may include every amino acid residue of the polypeptide.

As used herein, the terms “fragment” and “segment” can refer to a portion (e.g., at least 5, 10, 25, 50, 100, 125, 150, 200, 250, 300, 350, 400 or 500, etc. amino acids or nucleic acids) of a peptide that is substantially identical to a reference peptide and retains the biological activity of the reference peptide. In some aspects, the fragment or portion of a peptide retains at least 50%, 75%, 80%, 85%, 90%, 95% or 99% of the biological activity of the reference peptide described herein. A fragment of a referenced peptide can be a continuous or contiguous portion of the referenced polypeptide (e.g., a fragment of a reference peptide that is ten amino acids long can be any 2-9 contiguous residues within that reference peptide).

“Mutants,” “derivatives,” and “variants” of a polypeptide (or of the nucleic acid encoding the same) are polypeptides (or the nucleic acids) which may be modified or altered in one or more amino acids (or in one or more nucleotides) such that the peptide (or the nucleic acid) is not identical to the wild-type sequence, but has homology to the wild type polypeptide (or the nucleic acid).

The term “variant” can refer to a peptide or gene product that displays modifications in sequence and/or functional properties (i.e., altered characteristics) when compared to the wild-type peptide or gene product. In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level. In some aspects, the term “variant” can mean a difference in some way from the reference sequence other than just a simple deletion of an N- and/or C-terminal amino acid residue or residues. In an aspect, a variant can include a substitution of an amino acid residue, the substitution can be considered conservative or non-conservative. Conservative substitutions are those within the following groups: Ser, Thr, and Cys; Leu, lie, and Val; Glu and Asp; Lys and Arg; Phe, Tyr, and Trp; and Gin, Asn, Glu, Asp, and His. Variants can include at least one substitution and/or at least one addition, there may also be at least one deletion. Variants can also include one or more non-naturally occurring residues. For example, they may include selenocysteine (e.g., seleno-L-cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH₂(CH₂)ₙCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and orithine is basic. Proline may be substituted with hydroxyproline and retain the conformation conferring properties of proline.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

As used herein, the term “stability” refers to storage stability (e.g., room-temperature stability) as well as in vivo stability. The foregoing protective group can protect the peptides described herein from the attack of protein cleavage enzymes in vivo.

The peptides and fragments thereof disclosed herein can also include functional equivalents of the peptides described herein. As used herein, the term “functional equivalents” can refer to amino acid sequence variants having an amino acid substitution, addition, or deletion in some of the amino acid sequence of the peptide while simultaneously having similar or improved biological activity, compared with the peptide as described herein. In some aspects, the amino acid substitution can be a conservative substitution. Examples of the naturally occurring amino acid conservative substitution include, for example, aliphatic amino acids (Gly, Ala, and Pro), hydrophobic amino acids (Ile, Leu, and Val), aromatic amino acids (Phe, Tyr, and Trp), acidic amino acids (Asp and Glu), basic amino acids (His, Lys, Arg, Gln, and Asn), and sulfur-containing amino acids (Cys and Met). In some aspects, the amino acid deletion can be located in a region that is not directly involved in the activity of the peptide disclosed herein.

In some aspects, the amino acid sequence of the peptides and fragments thereof disclosed herein can include a peptide sequence that has substantial identity to any of the sequences of the peptides disclosed herein. As used herein, the term “substantial identity” means that two amino acid sequences, when optimally aligned and then analyzed by an algorithm normally used in the art, such as BLAST, GAP, or BESTFIT, or by visual inspection, share at least about 60%, 70%, 80%, 85%, 90%, or 95% sequence identity. Methods of alignment for sequence comparison are known in the art.

In some aspects, the amino acid sequence of the peptides and fragments thereof disclosed herein can include a peptide sequence that has some degree of identity or homology to any of sequences of the peptides disclosed herein. The degree of identity can vary and be determined by methods known to one of ordinary skill in the art. The terms “homology” and “identity” each refer to sequence similarity between two polypeptide sequences. Homology and identity can each be determined by comparing a position in each sequence which can be aligned for purposes of comparison. When a position in the compared sequence is occupied by the same amino acid residue, then the polypeptides can be referred to as identical at that position; when the equivalent site is occupied by the same amino acid (e.g., identical) or a similar amino acid (e.g., similar in steric and/or electronic nature), then the molecules can be referred to as homologous at that position. A percentage of homology or identity between sequences is a function of the number of matching or homologous positions shared by the sequences. The peptides described herein can have at least or about 25%, 50%, 65%, 75%, 80%, 85%, 90%, 95%, 96%, 97%, 98%, or 99% identity or homology to the peptide or polypeptide, wherein the peptide or polypeptide is, for example, one or more of SEQ ID NOs: 1-8 or wherein the peptide or polypeptide is, for example, D-SA or L-SA, as further described herein.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including, matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compounds and compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Peptides

In one aspect, disclosed are peptides useful in preparing the disclosed polypeptides and/or the disclosed bi-specific polypeptides. Without wishing to be bound by theory, the disclosed peptides can be prepared by methods known to one of skill in the art and as described elsewhere herein. Exemplary peptides are illustrated in Table 1 below.

TABLE 1

| ID | Sequence |
|---|---|
| Fragment 1 | AEAGITGTWYNQLGSTFIVTAGADGALTGT |
| precursor | YES (SEQ ID NO: 1) |
| Fragment 1 | AEAGITGTWYNQLGSTFIVTAGADGALTGT |
| | YES-N₂H₄ (SEQ ID NO: 2) |
| Fragment 2 | AVGNAESRYVLTGRYDSAPATDGSGTALGW |
| precursor | TVAWKNNYRNAHS-N₂H₄ |
| | (SEQ ID NO: 3) |
| Fragment 2 | AVGNAESRYVLTGRYDSAPATDGSGTALGW |
| | TVAWKNNYRNAHS-N₂H₄ |
| | (SEQ ID NO: 4) |
| Fragment 3 | ATTWSGQYVGGAEARINTQWLLTSGTTEAN |
| | AWKSTLVGHDTFTKVKPSAAS |
| | (SEQ ID NO: 5). |
| Linker + | X_{HH}AEAGITGTWYNQLGSTFIVTAGADGALT |
| Fragment 1 | GTYES-N₂H₄ (SEQ ID NO: 6) |
| Solubilizing | KKKKKKX_{HH}AEAGITGTWYNQLGSTFIVTAG |
| residue + | ADGALTGTYES-N₂H₄ (SEQ ID NO: 7) |
| Fragment 1 | |

In various aspects, each amino acid in the amino acid sequence is a D-amino acid or each amino acid in the amino acid sequence is a L-amino acid. For example, in one aspect, each amino acid in the amino acid sequence is a D-amino acid. As would be understood by one of ordinary skill in the art, peptides in which each amino acid is a D-amino acid can be useful in, for example, preparing D-polypeptides (e.g., bi-specific polypeptides in the D-configuration). In a further aspect, each amino acid in the amino acid sequence is a L-amino acid. Peptides in which each amino acid is a L-amino acid can be useful in, for example, preparing L-polypeptides (e.g., bi-specific polypeptides in the L-configuration).

Thus, in various aspects, disclosed are peptides comprising an amino acid sequence of at least 90% identity to the amino acid sequence AEAGITGTWYNQLGSTFIVTAGADGALTGTYES (SEQ ID NO:1), wherein each amino acid in the amino acid sequence is a D-amino acid or wherein each amino acid in the amino acid sequence is a L-amino acid. In a further aspect, disclosed are peptides comprising an amino acid sequence of at least 90% identity to the amino acid sequence AEAGITGTWYNQLGSTFIVTAGADGALTGTYES-N₂H₄(SEQ ID NO:2), wherein each amino acid in the amino acid sequence is a D-amino acid or wherein each amino acid in the amino acid sequence is a L-amino acid.

In various aspects, disclosed are peptides comprising an amino acid sequence of at least 90% identity to the amino acid sequence AVGNAESRYVLTGRYDSAPATDGSGTALGWTVAWKNNYRNAHS (SEQ ID NO:3), wherein each amino acid in the amino acid sequence is a D-amino acid or wherein each amino acid in the amino acid sequence is a L-amino acid. In a further aspect, disclosed are peptides comprising an amino acid sequence of at least 90% identity to the amino acid sequence XHHAEAGITGTWYNQLGSTFIVTAGADGALTGTYES-N₂H₄(SEQ ID NO:4), wherein each amino acid in the amino acid sequence is a D-amino acid or wherein each amino acid in the amino acid sequence is a L-amino acid.

In various aspects, disclosed are peptides comprising an amino acid sequence of at least 90% identity to the amino acid sequence ATTWSGQYVGGAEARINTQWLLTSGTTEANAWKSTLVGHDTFTKVKPSAAS (SEQ ID NO:5), wherein each amino acid in the amino acid sequence is a D-amino acid or wherein each amino acid in the amino acid sequence is a L-amino acid.

In various aspects, disclosed are peptides comprising an amino acid sequence of at least 90% identity to the amino acid sequence XHHAEAGITGTWYNQLGSTFIVTAGADGALTGTYES-N₂H₄(SEQ ID NO:6), wherein each amino acid in the amino acid sequence is a D-amino acid or wherein each amino acid in the amino acid sequence is a L-amino acid, and wherein X_{H}H is a linker. In a further aspect, disclosed are peptides comprising an amino acid sequence of at least 90% identity to the amino acid sequence KKKKKKX_{HH}AEAGITGTWYNQLGSTFIVTAGADGALTGTYES-N₂H₄(SEQ ID NO:7), wherein each amino acid in the amino acid sequence is a D-amino acid or wherein each amino acid in the amino acid sequence is a L-amino acid, and wherein KKKKKK and X_{HH} together comprise a solubilizing residue.

### C. Polypeptides

In one aspect, disclosed are polypeptides useful in preparing the disclosed bi-specific polypeptides. Without wishing to be bound by theory, the disclosed peptides can be prepared by methods described elsewhere herein. Exemplary polypeptides are illustrated in Table 2 below.

TABLE 2

| ID | Sequence |
|---|---|
| Fragment 1 + | AEAGITGTWYNQLGSTFIVTAGADGAL |
| Fragment 2 + | TGTYESAVGNAESRYVLTGRYDSAPAT |
| Fragment 3 | DGSGTALGWTVAWKNNYRNAHSATTWS |
| | GQYVGGAEARINTQWLLTSGTTEANAW |
| | KSTLVGHDTFTKVKPSAAS |
| | (SEQ ID NO: 8) |
| Solubilizing | KKKKKKX_{IIII}AEAGITGTWYNQLGSTF |
| residue + | IVTAGADGALTGTYESAVGNAESRYVL |
| Fragment 1 + | TGRYDSAPATDGSGTALGWTVAWKNNY |
| Fragment 2 | RNAHS-N₂H₄ (SEQ ID NO: 9). |
| Solubilizing | KKKKKKX_{HH}AEAGITGTWYNQLGSTFIV |
| residue + | TAGADGALTGTYESAVGNAESRYVLTG |
| Fragment 1 + | RYDSAPATDGSGTALGWTVAWKNNYRN |
| Fragment 2 + | AHSATTWSGQYVGGAEARINTQWLLTS |
| Fragment 3 | GTTEANAWKSTLVGHDTFTKVKPSAAS |
| | (SEQ ID NO: 10). |

In various aspects, each amino acid in the amino acid sequence is a D-amino acid or each amino acid in the amino acid sequence is a L-amino acid. For example, in one aspect, each amino acid in the amino acid sequence is a D-amino acid. As would be understood by one of ordinary skill in the art, polypeptides in which each amino acid is a D-amino acid can be useful in, for example, preparing bi-specific polypeptides in the D-configuration. In a further aspect, each amino acid in the amino acid sequence is a L-amino acid. Polypeptides in which each amino acid is a L-amino acid can be useful in, for example, preparing bi-specific polypeptides in the L-configuration.

Thus, in one aspect, disclosed are polypeptides comprising an amino acid sequence that has from 90% to 99% identity to the amino acid sequence AEAGITGTWYNQLGSTFIVTAGADGALTGTYES AVGNAESRYVLTGRYDSAPATDGSGTALGWTVAWKNNYRNAHS ATTWSGQYVGGAEARINTQWLLTSGTTEANAWKSTLVGHDTFTKVKPSAAS (SEQ ID NO:8), wherein each amino acid in the amino acid sequence is a D-amino acid.

In one aspect, disclosed are polypeptides comprising an amino acid sequence of at least 90% identity to the amino acid sequence KKKKKKX_{HH}AEAGITGTWYNQLGSTFIVTAGADGALTGTYES AVGNAESRYVLTGRYDSAPATDGSGTALGWTVAWKNNYRNAHS-N₂H₄(SEQ ID NO:9), wherein each amino acid in the amino acid sequence is a D-amino acid or wherein each amino acid in the amino acid sequence is a L-amino acid.

In one aspect, disclosed are polypeptides comprising an amino acid sequence of at least 90% identity to the amino acid sequence KKKKKKX_{HH}AEAGITGTWYNQLGSTFIVTAGADGALTGTYES AVGNAESRYVLTGRYDSAPATDGSGTALGWTVAWKNNYRNAHS ATTWSGQYVGGAEARINTQWLLTSGTTEANAWKSTLVGHDTFTKVKPSAAS (SEQ ID NO:10), wherein each amino acid in the amino acid sequence is a D-amino acid or wherein each amino acid in the amino acid sequence is a L-amino acid.

In various aspects, the polypeptide is D-traptavidin, D-strep-tactin, D-strep-tactin XT, or monovalent D-SA.

### D. Methods of Making a Polypeptide

In one aspect, disclosed are methods of making L-SA, D-SA, or a variant thereof, the method: (a) providing a first peptide having an amino acid sequence that has at least 90% identity to the amino acid sequence AEAGITGTWYNQLGSTFIVTAGADGALTGTYES-N₂H₄(SEQ ID NO:2), wherein each amino acid in the amino acid sequence is a D-amino acid or wherein each amino acid in the amino acid sequence is a L-amino acid; (b) providing a second peptide having an amino acid sequence that has at least 90% identity to the amino acid sequence AVGNAESRYVLTGRYDSAPATDGSGTALGWTVAWKNNYRNAHS-N₂H₄(SEQ ID NO:4), wherein each amino acid in the amino acid sequence is a D-amino acid or wherein each amino acid in the amino acid sequence is a L-amino acid; (c) providing a third peptide having an amino acid sequence that has at least 90% identity to the amino acid sequence ATTWSGQYVGGAEARINTQWLLTSGTTEANAWKSTLVGHDTFTKVKPSAAS (SEQ ID NO:5), wherein each amino acid in the amino acid sequence is a D-amino acid or wherein each amino acid in the amino acid sequence is a L-amino acid; and (d) ligating the first, second, and third peptides.

In one aspect, disclosed are methods of making L-SA, D-SA, or a variant thereof, the method comprising: (a) coupling a first peptide having an amino acid sequence that has at least 90% identity to the amino acid sequence AEAGITGTWYNQLGSTFIVTAGADGALTGTYES-N₂H₄(SEQ ID NO:2) and a linker, wherein each amino acid in the amino acid sequence is a D-amino acid or wherein each amino acid in the amino acid sequence is a L-amino acid; (b) functionalizing the linker with a positively charged amino acid residue, thereby providing a solubilizing residue; (c) ligating the first peptide to a second peptide having an amino acid sequence that has at least 90% identity to the amino acid sequence AVGNAESRYVLTGRYDSAPATDGSGTALGWTVAWKNNYRNAHS-N₂H₄(SEQ ID NO:4), wherein each amino acid in the amino acid sequence is a D-amino acid or wherein each amino acid in the amino acid sequence is a L-amino acid; (d) ligating the second peptide to a third peptide having an amino acid sequence that has at least 90% identity to the amino acid sequence ATTWSGQYVGGAEARINTQWLLTSGTTEANAWKSTLVGHDTFTKVKPSAAS (SEQ ID NO:5), wherein each amino acid in the amino acid sequence is a D-amino acid or wherein each amino acid in the amino acid sequence is a L-amino acid; and (e) cleaving the solubilizing residue from the polypeptide.

In one aspect, disclosed are methods of making a polypeptide comprising an amino acid sequence that has at least 90% identity to the amino acid sequence KKKKKKX_{HH}AEAGITGTWYNQLGSTFIVTAGADGALTGTYES AVGNAESRYVLTGRYDSAPATDGSGTALGWTVAWKNNYRNAHS ATTWSGQYVGGAEARINTQWLLTSGTTEANAWKSTLVGHDTFTKVKPSAAS (SEQ ID NO:10), wherein each amino acid in the amino acid sequence is a D-amino acid or wherein each amino acid in the amino acid sequence is a L-amino acid, the method comprising: (a) providing a first polypeptide having an amino acid sequence that has at least 90% identity to the amino acid sequence KKKKKKX_{HH}AEAGITGTWYNQLGSTFIVTAGADGALTGTYES AVGNAESRYVLTGRYDSAPATDGSGTALGWTVAWKNNYRNAHS-N₂H₄(SEQ ID NO:9), wherein each amino acid in the amino acid sequence is a D-amino acid or wherein each amino acid in the amino acid sequence is a L-amino acid; (b) providing a third peptide having an amino acid sequence that has at least 90% identity to the amino acid sequence ATTWSGQYVGGAEARINTQWLLTSGTTEANAWKSTLVGHDTFTKVKPSAAS (SEQ ID NO:5), wherein each amino acid in the amino acid sequence is a D-amino acid or wherein each amino acid in the amino acid sequence is a L-amino acid; and (c) ligating the first polypeptide to the third peptide.

In one aspect, disclosed are methods of making L-SA, D-SA, or a variant thereof, the method comprising: (a) providing a polypeptide having an amino acid sequence that has at least 90% identity to the amino acid sequence KKKKKKX_{HH}AEAGITGTWYNQLGSTFIVTAGADGALTGTYES AVGNAESRYVLTGRYDSAPATDGSGTALGWTVAWKNNYRNAHS ATTWSGQYVGGAEARINTQWLLTSGTTEANAWKSTLVGHDTFTKVKPSAAS (SEQ ID NO:10), wherein each amino acid in the amino acid sequence is a D-amino acid or wherein each amino acid in the amino acid sequence is a L-amino acid, and wherein KKKKKK and X_{H}H together comprise a solubilizing residue; and (b) cleaving the solubilizing residue from the polypeptide.

In various aspects, the method further comprises coupling a linker to the first peptide prior to the ligating step. As used herein, the term “linker” refers to a bifunctional traceless linker that can be used, for example, to temporarily attach highly solubilizing peptide sequences (e.g., sequences of lysine residues) onto a peptide (e.g., an insoluble peptide). See, e.g., Jacobsen et al. (2016) JACS 138: 11775-11782. Once these highly solubilizing peptide sequences are attached, the linker in combination with the highly solubilizing peptide sequences form a solubilizing residue, which can be subsequently removed from the peptide.

Thus, in various aspects, the linker has a structure represented by a formula:

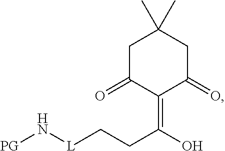

wherein L is a linker; and PG is an amine protecting group. Exemplary linkers include, but are not limited to, alkyl and alkoxy residues. As would be readily understood by one of ordinary skill in the art, an amine protecting group can be used in, for example, peptide synthesis, to enable other functional groups on the peptide to undergo selective reactions with electrophiles whereby the protected amine does not. The protecting group can then be subsequently removed. Exemplary amine protecting groups include, but are not limited to, 9-fluoroenylmethyl carbamate (Fmoc), tert-butyl carbamate (Boc), benzyl carbamate (Cbz), acetamide (Ac), trifluoroacetamide, and phthalimide.

Thus, in various aspects, the linker is selected from C1-C12 alkyl and —(CH₂CH₂O)ₙ—, wherein n is selected from 1, 2, 3, and 4. In a further aspect, the linker is a C1-C12 alkyl. In a still further aspect, the linker is a C1-C8 alkyl. In yet a further aspect, the linker is a C1-C4 alkyl (e.g., —CH₂—, —CH₂CH₂—, —CH₂CH₂CH₂—, —CH(CH₃)CH₂—, etc.). In an even further aspect, the linker is a C4 alkyl.

In a further aspect, the linker is —(CH₂CH₂O)ₙ—, wherein n is selected from 1, 2, 3, and 4 (e.g., —CH₂CH₂O—, —(CH₂CH₂O)₂—, —(CH₂CH₂O)₃—, etc.). In a still further aspect, n is selected from 1, 2, and 3. In yet a further aspect, n is selected from 1 and 2. In an even further aspect, n is 1. In a still further aspect, n is 2.

In various aspects, the amine protecting group is selected from 9-fluoroenylmethyl carbamate (Fmoc), tert-butyl carbamate (Boc), benzyl carbamate (Cbz), acetamide (Ac), trifluoroacetamide, and phthalimide. In a further aspect, the amine protecting group is Fmoc.

In a further aspect, the linker has a structure:

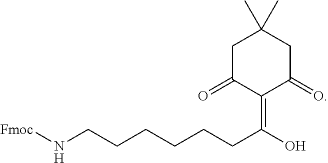

In a further aspect, the method further comprises deprotecting the linker prior to the ligating step. For example, as illustrated above, a deprotected linker can include:

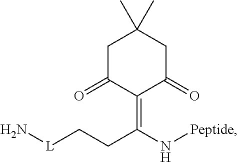

wherein the peptide is, for example, Fragment 1 (SEQ ID NO:2). Thus, in various aspects, the method comprises preparing a peptide as shown in SEQ ID NO:6, which can then be subsequently ligated to additional peptide fragments (e.g., Fragment 2 (SEQ ID NO:4) and Fragment 3 (SEQ ID NO:5)).

In a further aspect, the method further comprises functionalizing the linker with a positively charged amino acid residue prior to the ligating step. In a still further aspect, the positively charged amino acid residue is a lysine. In yet a further aspect, the free amine is functionalized with more than one positively charged amino acid residue (e.g., one, two, three, four, five, siz, seven, eight positively charged amino acid residues), wherein the positively charged amino acid residues are the same or different. In an even further aspect, each positively charged amino acid residue is lysine. In a still further aspect, the free amine is functionalized with six lysine residues. For example, as illustrated above, a deprotected linker functionalized with a positively charged amino acid residue (i.e., a solubilizing residue) can include:

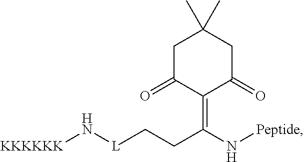

wherein the peptide is, for example, Fragment 1 (SEQ ID NO:2). Thus, in various aspects, the method comprises preparing a peptide as shown in SEQ ID NO:7, which can then be subsequently ligated to additional peptide fragments (e.g., Fragment 2 (SEQ ID NO:4) and Fragment 3 (SEQ ID NO:5)).

In a further aspect, the first peptide is ligated to the second peptide prior to ligating to the third peptide. Thus, in various aspects, the method comprises preparing a polypeptide as shown in SEQ ID NO:9, which can then be subsequently ligated to additional peptide fragments (e.g., Fragment 3 (SEQ ID NO:5)).

In a further aspect, the method further comprising cleaving the solubilizing residue. See, e.g., SEQ ID NO:8.

In a further aspect, ligating is via with native chemical ligation or any other ligation chemistry. In a still further aspect, ligating is via transthio-esterification. I

In a further aspect, the method makes L-SA or a variant thereof (e.g., L-traptavidin, monovalent L-SA.).

In a further aspect, the method makes D-SA or a variant thereof (e.g., D-traptavidin, monovalent D-SA.).

### E. Bi-Specific Polypeptides D-Sa or a Variant Thereof

In one aspect, disclosed are bi-specific polypeptides comprising a single chain antibody (scFv) or a F′ab fragment and a disclosed polypeptide. In a further aspect, the polypeptide comprises an amino acid sequence that has from 90% to 99% identity to the amino acid sequence AEAGITGTWYNQLGSTFIVTAGADGALTGTYES AVGNAESRYVLTGRYDSAPATDGSGTALGWTVAWKNNYRNAHS ATTWSGQYVGGAEARINTQWLLTSGTTEANAWKSTLVGHDTFTKVKPSAAS (SEQ ID NO:8), wherein each amino acid in the amino acid sequence is a D-amino acid. In a still further aspect, the scFv is an anti-CD30 antibody. In yet a further aspect, the scFv is 5F11 scFv.

In one aspect, disclosed are bi-specific polypeptides comprising an antibody or antibody fragment thereof covalently linked to D-SA or a variant thereof.

In various aspects, the antibody or antibody fragment thereof is an anti-IL-17 receptor antibody, an anti-IL-5 receptor antibody, an anti-PD-L1 antibody, an anti-FGF23 antibody, an anti-epithelial growth factor receptor antibody, an anti-GD2 antibody, an anti-HER-2 receptor antibody, an anti-RANKL antibody, an anti-C5 antibody, an anti-VEGF receptor antibody, an anti-VEGF-A antibody, an anti-VEGF receptor-2 antibody, anti-IgE antibody, an anti-TNF-alpha antibody, an anti-IL-12/23 antibody, an anti-CTLA-4 antibody, an anti-CD30 antibody, an anti-CD4 antibody, an anti-CGRP receptor antibody, an anti-CD3 antibody, an anti-CD20 antibody, an anti-CD25 antibody, or an anti-GP11b/llla antibody. In a further aspect, the antibody or antibody fragment thereof is an anti-CD30 antibody.

In various aspects, the antibody or antibody fragment thereof specifically binds a cell surface marker. In a further aspect, the cell surface marker is CD3, CD4, CD5, CD20, CD25, or a glycosphingolipid. In a still further aspect, the glycosphingolipid is GD₂.

In various aspects, the antibody or antibody fragment thereof is a single chain antibody (scFv) or a F′ab fragment. In a still further aspect, the scFv is derived from burosumab, ibalizumab, erenumab, atezolizumab, reslizumab, pembrolizumab, nivolumab, ramucirumab, ipilimumab, brentuximab, ustekinumab, panitumaumab, ranibizumab, necitumaumab, dinutuximab, denosumab, exulizumab, bevacizumab, omalizumab, adalimumab, avelumab, durvalumab, brodalumab, muromonoab-CD3, abciximab, rituximab, daclizumab, infliximab, basiliximab, palivizumab, trastuzumab, gemtuzumab, or a biologically active variant thereof.

In various aspects, the antibody or antibody fragment thereof is covalently linked to D-SA.

In various aspects, the antibody or antibody fragment thereof is covalently linked to a variant of D-SA. In a further aspect, the variant of D-SA is D-traptavidin, D-strep-tactin, D-strep-tactin XT, or monovalent D-SA.

In various aspects, the D-SA or variant thereof specifically binds D-biotin.

In various aspects, the bi-specific polypeptide further comprises a label or detection tag. In various further aspects, the label or detection tag is a sortase tag.

### 1. Labels

In various aspects, the bi-specific polypeptides and compositions described herein can further comprise one or more labels or detection tags (e.g., FLAG™ tag, epitope or protein tags, such as myc tag, 6 His, and fluorescent fusion protein). In various aspects, the label (e.g., FLAG™ tag) can be fused to a component of the disclosed bi-specific polypeptide (e.g., a bi-specific polypeptide comprising D-SA or a variant thereof). In various aspects, the disclosed methods and compositions further comprise a fusion protein, or a polynucleotide encoding the same. In various aspects, the bi-specific polypeptide or fusion protein comprises at least one epitope-providing amino acid sequence (e.g., “epitope-tag”), wherein the epitope-tag is selected from i) an epitope-tag added to the N- and/or C-terminus of the protein (e.g., D-SA or a variant thereof); or ii) an epitope-tag inserted into a region of the protein (e.g., D-SA or a variant thereof), and an epitope-tag replacing a number of amino acids in the protein (e.g., D-SA or a variant thereof).

In various aspects, the bi-specific polypeptides and compositions described herein can further comprise a poly glycine and a sortase tag (i.e., Leu-Pro-Xxx-Thr-Gly-Xxx, where Xxx is any amino acid). Such a tag can be useful in, for example, performing a sortase ligation (e.g., between D-SA and a scFv).

As would be understood by one of ordinary skill in the art, a tag can be installed at the N-terminus or the C-terminus of a peptide, although smaller tags can be installed at most any positions within synthetic proteins (e.g., D-SA).

Epitope tags are short stretches of amino acids to which a specific antibody can be raised, which In various aspects allows one to specifically identify and track the tagged protein that has been added to a living organism or to cultured cells. Detection of the tagged molecule can be achieved using a number of different techniques. Examples of such techniques include: immunohistochemistry, immunoprecipitation, flow cytometry, immunofluorescence microscopy, ELISA, immunoblotting (“Western blotting”), and affinity chromatography. Epitope tags add a known epitope (e.g., antibody binding site) on the subject protein, to provide binding of a known and often high-affinity antibody, and thereby allowing one to specifically identify and track the tagged protein that has been added to a living organism or to cultured cells. Examples of epitope tags include, but are not limited to, myc, T7, GST, GFP, HA (hemagglutinin), V5 and FLAG tags. The first four examples are epitopes derived from existing molecules. In contrast, FLAG is a synthetic epitope tag designed for high antigenicity (see, e.g., U.S. Pat. Nos. 4,703,004 and 4,851,341). Epitope tags can have one or more additional functions, beyond recognition by an antibody.

In various aspects, the disclosed methods, bi-specific polypeptides, and compositions comprise an epitope-tag wherein the epitope-tag has a length of between 6 to 15 amino acids. In an alternative aspect, the epitope-tag has a length of 9 to 11 amino acids. The disclose methods and compositions can also comprise a bi-specific polypeptide comprising two or more epitope-tags, either spaced apart or directly in tandem. Further, the disclosed methods and bi-specific polypeptides or compositions can comprise 2, 3, 4, 5 or even more epitope-tags, as long as the bi-specific polypeptide maintains its biological activity/activities (e.g., “functional”).

In various aspects, the epitope-tag can be a VSV-G tag, CD tag, calmodulin-binding peptide tag, S-tag, Avitag, SF-TAP-tag, strep-tag, myc-tag, FLAG-tag, T7-tag, HA (hemagglutinin)-tag, His-tag, GST-tag, or GFP-tag. The sequences of these tags are described in the literature and are well known to the person of skill in art.

As described herein, the term “immunologically binding” is a non-covalent form of attachment between an epitope of an antigen (e.g., the epitope-tag) and the antigen-specific part of an antibody or fragment thereof. Antibodies are preferably monoclonal and must be specific for the respective epitope tag(s) as used. Antibodies include murine, human and humanized antibodies. Antibody fragments are known to the person of skill and include, amongst others, single chain Fv antibody fragments (scFv fragments) and Fab-fragments. The antibodies can be produced by regular hybridoma and/or other recombinant techniques. Many antibodies are commercially available.

The construction of bi-specific polypeptides from domains of known proteins, or from whole proteins or proteins and peptides, is well known. Generally, a nucleic acid molecule that encodes the desired protein and/or peptide portions are joined using genetic engineering techniques to create a single, operably linked fusion oligonucleotide. Appropriate molecular biological techniques can be found in Sambrook et al. (Molecular Cloning: A laboratory manual Second Edition Cold Spring Harbor Laboratory Press, Cold spring harbor, NY, USA, 1989). Examples of genetically engineered multi-domain proteins, including those joined by various linkers, and those containing peptide tags, can be found in the following patent documents: U.S. Pat. No. 5,994,104 (“Interleukin-12 fusion protein”); U.S. Pat. No. 5,981,177 (“Protein fusion method and construction”); U.S. Pat. No. 5,914,254 (“Expression of fusion polypeptides transported out of the cytoplasm without leader sequences”); U.S. Pat. No. 5,856,456 (“Linker for linked fusion polypeptides”); U.S. Pat. No. 5,767,260 (“Antigen-binding fusion proteins”); U.S. Pat. No. 5,696,237 (“Recombinant antibody-toxin fusion protein”); U.S. Pat. No. 5,587,455 (“Cytotoxic agent against specific virus infection”); U.S. Pat. No. 4,851,341 (“Immunoaffinity purification system”); U.S. Pat. No. 4,703,004 (“Synthesis of protein with an identification peptide”); and WO 98/36087 (“Immunological tolerance to HIV epitopes”).

The placement of the functionalizing peptide portion (epitope-tag) within the subject fusion proteins can be influenced by the activity of the functionalizing peptide portion and the need to maintain at least substantial fusion protein, such as TCR, biological activity in the fusion. Two methods for placement of a functionalizing peptide are: N-terminal, and at a location within a protein portion that exhibits amenability to insertions. Though these are not the only locations in which functionalizing peptides can be inserted, they serve as good examples, and will be used as illustrations. Other appropriate insertion locations can be identified by inserting test peptide encoding sequences (e.g., a sequence encoding the FLAG peptide) into a construct at different locations, then assaying the resultant fusion for the appropriate biological activity and functionalizing peptide activity, using assays that are appropriate for the specific portions used to construct the fusion. The activity of the subject proteins can be measured using any of various known techniques, including those described herein.

### F. Conjugates Comprising L-Biotin

In one aspect, disclosed are conjugates comprising L-biotin covalently linked to a therapeutic agent or a diagnostic agent.

In various aspects, the conjugate is covalently linked to a therapeutic agent. In a further aspect, the therapeutic agent is a chemotherapeutic agent. Examples of chemotherapeutic agents include, but are not limited to doxorubicin, cisplatin, 5-fluorouracin (5-FU), etoposide, daunorubicin, camptothesin, methotrexate, carboplatin, and oxaliplatin.

In various aspects, the conjugate is covalently linked to a diagnostic agent. In a further aspect, the diagnostic agent is a cancer diagnostic agent.

### G. Pharmaceutical Compositions

In one aspect, disclosed are pharmaceutical compositions comprising an effective amount of a disclosed bi-specific polypeptide and a pharmaceutically acceptable carrier. In a further aspects, the pharmaceutical composition comprises an effective amount of a composition comprising a disclosed bi-specific polypeptide and a pharmaceutically acceptable carrier.

In various aspects, the bi-specific polypeptide comprises a single chain antibody (scFv) or a F′ab fragment and a polypeptide comprising an amino acid sequence that has from 90% to 99% identity to the amino acid sequence AEAGITGTWYNQLGSTFIVTAGADGALTGTYES AVGNAESRYVLTGRYDSAPATDGSGTALGWTVAWKNNYRNAHS ATTWSGQYVGGAEARINTQWLLTSGTTEANAWKSTLVGHDTFTKVKPSAAS (SEQ ID NO:8), wherein each amino acid in the amino acid sequence is a D-amino acid. In various further aspects, the bi-specific polypeptide comprises an antibody or antibody fragment thereof covalently linked to D-SA or a variant thereof.

In various aspects, the bi-specific polypeptide comprises L-biotin covalently linked to a therapeutic agent or a diagnostic agent.

The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used to deliver the nanoparticles. Thus, compositions can be prepared for parenteral administration that includes nanoparticles dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

In various aspects, the pharmaceutical composition is formulated for intravenous administration.

### H. Methods of Treating a Disease or a Condition

In one aspect, disclosed are methods of treating a disease or a condition in a subject in need thereof, the method comprising: (a) administering to the subject an effective amount of a disclosed bi-specific polypeptide; and (b) subsequently administering to the subject an effective amount of a composition comprising L-biotin covalently linked to a therapeutic agent, wherein the D-SA or the variant thereof specifically binds L-biotin.

As would be understood by one of ordinary skill in the art, bi-specific polypeptides comprising other proteins that bind biotin with a high affinity (i.e., proteins other than streptavidin) can also be used in the disclosed methods. Thus, for example, in various aspects, avidin can be used in place of the streptavidin (e.g., L-avidin, D-avidin).

Any of the compositions described herein can be administered as a “combination.”

The pharmaceutical compositions described herein can be administered to the subject (e.g., a human patient) in an amount sufficient to delay, reduce, or preferably prevent the onset of clinical disease. Accordingly, In various aspects, the patient can be a human patient. In therapeutic applications, compositions can be administered to a subject (e.g., a human patient) already with or diagnosed with cancer (or autoimmune disease or disorder) in an amount sufficient to at least partially improve a sign or symptom or to inhibit the progression of (and preferably arrest) the symptoms of the condition, its complications, and consequences. In various aspects, compositions can be administered to a subject (e.g., a human patient) already with or diagnosed with cancer (or an autoimmune disease or disorder). In various aspects, compositions can be administered to a subject (e.g., a human patient) already with or diagnosed with cancer and with or diagnosed with an autoimmune disease or disorder in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a pharmaceutical composition can be an amount that achieves a cure, but that outcome is only one among several that can be achieved. As noted, a therapeutically effect amount includes amounts that provide a treatment in which the onset or progression of the cancer (or autoimmune disease or disorder) is delayed, hindered, or prevented, or a symptom of the cancer (or autoimmune disease or disorder) is ameliorated. One or more of the symptoms can be less severe. Recovery can be accelerated in an individual who has been treated.

Amounts effective for this use can depend on the severity of the cancer and the weight and general state and health of the subject, but generally range from about 0.1 mg/kg body to about 10.0 mg/kg body weight per dose per subject. Suitable regimes for initial administration and booster administrations are typified by an initial administration followed by repeated doses at one or more hourly, daily, weekly, or monthly intervals by a subsequent administration. For example, a subject can receive nanoparticles in the range of about 0.1 mg/kg body weight to about 10 mg/kg body weight per dose one or more times per week (e.g., 2, 3, 4, 5, 6, or 7 or more times per week). For example, a subject can receive 0.1 mg/kg body weight to 10 mg/kg body weight (e.g., 0.3, 1.0, 3.0, 10.0 mg/kg body weight) dose per week. A subject can also receive nanoparticles in the range of 0.1 mg/kg body weight to 10 mg/Kg body weight per dose once every two or three weeks. The total effective amount of an nanoparticles in the pharmaceutical compositions disclosed herein can be administered to a mammal as a single dose, either as a bolus or by infusion over a relatively short period of time, or can be administered using a fractionated treatment protocol in which multiple doses are administered over a more prolonged period of time (e.g., a dose every 4-6, 8-12, 14-16, or 18-24 hours, or every 2-4 days, 1-2 weeks, or once a month). Alternatively, continuous intravenous infusions sufficient to maintain therapeutically effective concentrations in the blood are also within the scope of the present disclosure.

The therapeutically effective amount of one or more of the therapeutic agents present within the compositions described herein and used in the methods as disclosed herein applied to mammals (e.g., humans) can be determined by one of ordinary skill in the art with consideration of individual differences in age, weight, and other general conditions (as mentioned above).

The combination therapies disclosed herein can be administered as one or more pharmaceutical compositions and, if separately, can be administered simultaneously or sequentially in any order.

In various aspects, the compositions can include a mixture of two or more such compounds in equal or unequal amounts.

The particular combination of agents can vary according to many factors, for example, the particular kind of cancer, the severity of the cancer, any comorbidities, and the health of the patient.

When a combination of any of the compositions disclosed herein is administered to the same patient, they can be administered in a single formulation (e.g., a co-formulation) or in separate formulations (which may be the same or different) that can be administered concurrently or sequentially.

In various aspects, the effective amount is a therapeutically effective amount. In various further aspect, the effective amount is a prophylactically effective amount.

In various aspects, the subject is a mammal. In various further aspects, the subject is a human.

In various aspects, the subject has been diagnosed with a need for treatment of the disease or the condition prior to the administering step. In various further aspects, the method further comprises the step of identifying a subject in need of treatment of the disease or the condition.

In various aspects, the method treats a disease. Exemplary diseases include, but are not limited to, cancer, non-Hodgkin lymphoma, multiple sclerosis, Crohn's disease, rheumatoid arthritis, asthma, macular degeneration, psoriasis, Hodgkin lymphoma, paroxysmal nocturnal hemoglobinuria, and X-linked hypophosphatemia. In a further aspect, the disease is cancer. In a still further aspect, the cancer is a primary or secondary cancer. In yet a further aspect, the primary or secondary cancer is a sarcoma, a carcinoma, brain cancer, breast cancer, renal cancer, pancreatic cancer, lung cancer, liver cancer, lymphoma, prostate cancer, colon cancer, ovarian cancer, gastrointestinal cancer, colorectal cancer, skin cancer. thyroid cancer, testicular cancer, endometrial cancer, melanoma, a glioma, leukemia, neuroblastoma, cervical cancer, chronic myeloproliferative disorder, myelodysplastic syndrome, a hematological cancer, myeloproliferative neoplasm, non-small cell lung carcinoma, gastroesophageal junction cancer, bladder cancer, Merkel cell carcinoma, urothelial carcinoma, or plasma cell neoplasm (myeloma). In an even further aspect, the cancer is neuroblastoma.

In various aspects, the method treats a condition. Exemplary conditions include, but are not limited to, prevention of blood clots in angioplasty, kidney transplantation rejection, migraine prevention, HIV infection, and bone loss.

In various aspects, the therapeutic agent is administered at least 1, 2, 3, 4, 5, 6, 7, 8, 9, 10, 11, 12, 13, 14, 15, 16, 17, 18, 19, 20, 21, 22, 23, or 24 hour(s) subsequent to the bi-specific polypeptide. In a further aspect, the therapeutic agent is administered 24 hours subsequent to the bi-specific polypeptide. In a still further aspect, the therapeutic agent is administered 48 hours subsequent to the bi-specific polypeptide. In yet a further aspect, the therapeutic agent is administered 1 week subsequent to the bi-specific polypeptide. In an even further aspect, the therapeutic agent is administered more than 1 week subsequent to the bi-specific polypeptide.

In various aspects, the therapeutic agent is a chemotherapeutic agent.

Examples of chemotherapeutic agents include, but are not limited to, doxorubicin, cisplatin, 5-fluorouracin (5-FU), etoposide, daunorubicin, camptothesin, methotrexate, carboplatin, and oxaliplatin.

In various aspects, the D-SA or the variant thereof specifically binds L-biotin.

### I. Kits

In one aspect, disclosed are kits comprising a disclosed bi-specific polypeptide, and one or more selected from: (a) a therapeutic agent; (b) L-biotin; (c) instructions for administering the bi-specific polypeptide in connection with treating a disease or a condition; and (d) instructions for treating the disease or the condition.

In various aspects, the therapeutic agent is a chemotherapeutic agent.

Examples of chemotherapeutic agents include, but are not limited to, alkylating agents, antimetabolite agents, antineoplastic antibiotic agents, mitotic inhibitor agents, and mTor inhibitor agents.

In various aspects, the chemotherapeutic agent is an antineoplastic agent. In a further aspect, the antineoplastic antibiotic agent is selected from doxorubicin, mitoxantrone, bleomycin, daunorubicin, dactinomycin, epirubicin, idarubicin, plicamycin, mitomycin, pentostatin, and vairubicin, or a pharmaceutically acceptable salt thereof.

In various aspects, the chemotherapeutic agent is an antimetabolite agent. In a further aspect, the antimetabolite agent is selected from gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof.

In various aspects, the chemotherapeutic agent is an alkylating agent. In a further aspect, the alkylating agent is selected from carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt thereof.

In various aspects, the chemotherapeutic agent is a mitotic inhibitor agent. In a still further aspect, the mitotic inhibitor agent is selected from irinotecan, topotecan, rubitecan, cabazitaxel, docetaxel, paclitaxel, etopside, vincristine, ixabepilone, vinorelbine, vinblastine, and teniposide, or a pharmaceutically acceptable salt thereof.

In various aspects, the chemotherapeutic agent is a mTor inhibitor agent. In a still further aspect, the mTor inhibitor agent is selected from everolimus, siroliumus, and temsirolimus, or a pharmaceutically acceptable salt thereof.

In various aspects, the therapeutic agent is covalently linked to L-biotin.

In various aspects, the bi-specific polypeptide and the therapeutic agent are co-packaged. In various further aspects, the bi-specific polypeptide and the therapeutic agent are co-formulated.

In various aspects, the disease is cancer.

### J. Examples

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

### 1. Proposed Strategy

As detailed herein, mirror-image SA and biotin (D-SA/L-biotin) offer an elegant solution to circumvent the limitations encountered with L-SA/D-biotin systems. For example, D-proteins are inert to L-proteases and therefore cannot be digested for MHC presentation to the immune system. This property means that D-SA will have greatly decreased immunogenicity and increased half-life compared to L-SA. Additionally, symmetry dictates that the mirror-image pair (D-SA/L-biotin) will have the exact same exceptional affinity as the natural pair (L-SA/D-biotin). Importantly, it has been disovered that D-biotin has minimal binding to D-SA. Therefore, it is proposed that D-SA/L-biotin can be used as a biotin orthogonal streptaviding system (BOSS). Without wishing to be bound by theory, it is hypothesized that the orthogonality of BOSS, along with D-SA's low immunogenicity, will overcome the limitations of natural SA/biotin.

BOSS can be created by using mirror-image SA and Biotin (D-SA and L-biotin). Mirror-image, or D-, proteins (composed of mirror-image D-amino acids) are inert to L-proteases²¹ and therefore cannot be digested for MHC presentation to the immune system.²² These properties mean that D-SA will have greatly decreased immunogenicity and increased half-life compared to L-SA. It is also true (by the law of mirror-image symmetry) that, since natural SA (L-SA) and natural biotin (D-biotin) have exceptional affinity, D-SA and L-biotin will have the exact same affinity. However, low immunogenicity, increased half-life, and a strong binding interaction between D-SA and L-biotin are not enough to create BOSS. For BOSS to be successful, it may be useful to demonstrate that natural D-biotin shows minimal binding to D-SA (i.e., orthogonality). The utility of mirror-image SA was recently reported in a short pre-print that sought to avoid biotin interference in immunoassay diagnostics.”

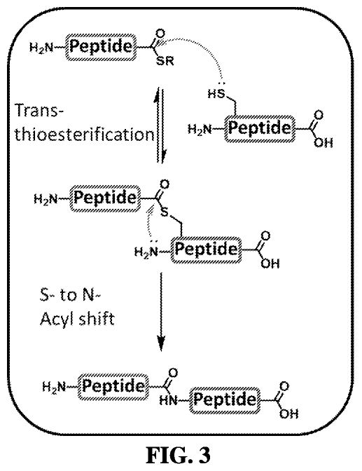

Since D-proteins cannot be produced recombinantly, D-SA will be chemically synthesized through chemical protein synthesis (CPS) using D-amino acids. A protein of this size (127 amino acids) requires synthesis in multiple segments (usually limited to ˜60 amino acids because of on-resin aggregation) via solid-phase peptide synthesis (SPPS).⁴¹ Native chemical ligation (NCL)⁴² is then used to link the segments together (FIG. 3). NCL requires one peptide with an N-terminal Cys and the other peptide with a C-terminal thioester. Thioester peptides are made using a C-terminal hydrazine on resin during SPPS. Post-cleavage conversion into an azide makes the hydrazine into a better leaving group, which can then be displaced by various thiols to make a thioester. NCL is then accomplished by reacting the C-terminal thioester with the N-terminal Cys on a separate segment. This reaction forms a reversible thioester bond between the two segments and brings the N-terminus close enough for an S- to N-acyl shift, forming an irreversible native amide bond (FIG. 3). Since many proteins (including SA) lack Cys residues, native N-terminal Ala residues can be replaced with cysteine residues for use in NCL. After the ligation, Cys can be converted to the native Ala with a simple desulfurization reaction.⁴¹ This strategy greatly increases the number of available ligation junctions.

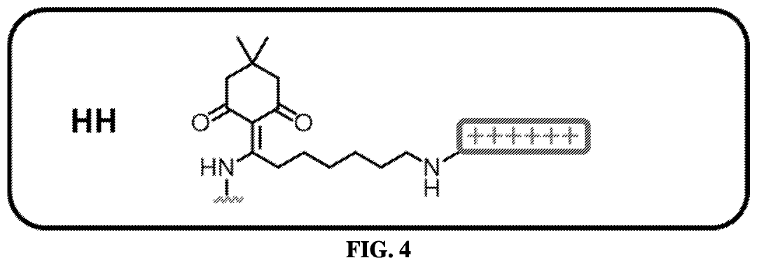

Proteins the size of SA have long been considered challenging to make by CPS. Recently, tools were developed that have made the synthesis of proteins of this size much more feasible. One of these tools is Automated Ligator (Aligator),²³ a program developed to predict optimal synthetic strategies, based on parameters that minimize the number of peptide segments and their length as well as optimizing ligation junctions. This program greatly reduces the trial and error in CPS, one of the biggest rate-limiting steps to synthesizing a protein. Additionally, peptide solubility can be a rate-limiting step. Breaking up proteins into segments often exposes regions with a high density of hydrophobic or negatively charged residues that lead to poor solubility, making purification of segments and ligations difficult. To combat insoluble peptides, traceless linkers that called helping hands (HH) were used. An HH^{44,45} that attaches to primary amines (FIG. 4), which can be functionalized with solubilizing residues (usually Lys or Arg). After the full protein is assembled and the solubility enhancement is no longer needed, the HHs are removed under gentle conditions to yield the native amino acid sidechain.

### 2. Characterization of L-Biotin and L-Streptavidin Binding

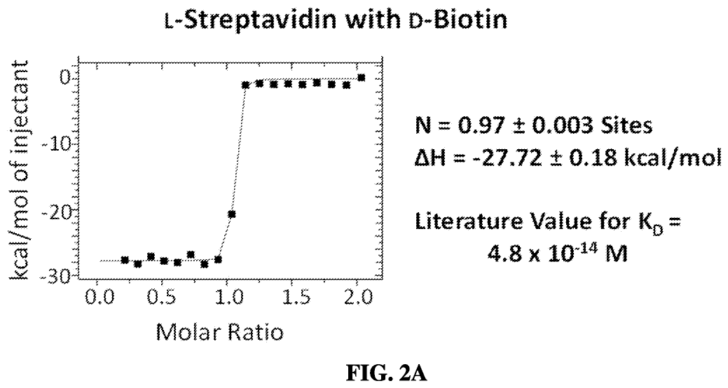

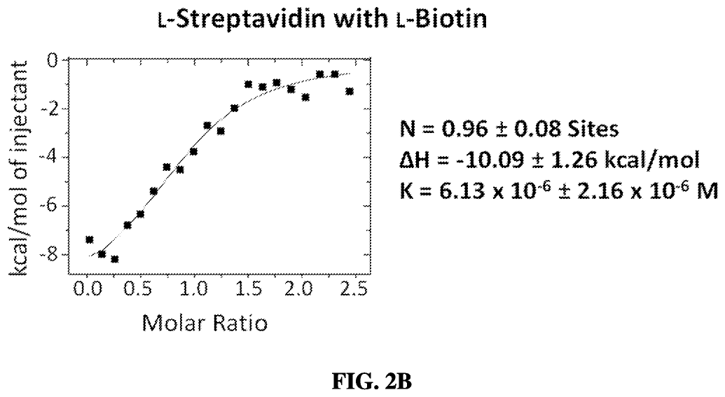

As tight cross-bonding would undermine the orthogonality of BOSS, the binding of D-biotin to D-streptavidin should be characterized. By the law of mirror-image symmetry, the binding behavior of L-biotin to L-streptavidin is equivalent to the D-biotin interaction. First, a control isothermal titration calorimetry (ITC) experiment was performed. D-biotin was titrated into L-SA to confirm folding and activity of the recombinant SA (Novus Biologicals). This titration data was fit using Origin software to calculate binding stoichiometry (N=0.97±0.003 sites) and enthalpy (ΔH=−27.72±0.18 kcal/mol), as shown in FIG. 2A. These values were consistent with literature values.^{27,39} The extraordinarily strong interaction between L-SA and D-biotin prevents determination of the K_{D} value from this experiment, but the binding constant has been measured using more sensitive techniques and found to be 4.8×104 M.¹

Next, the ITC experiment was repeated with L-biotin (Carbosynth) and L-SA. It was expected that this interaction would exhibit weaker binding to the hypothesized stereochemical mismatch between L-biotin and L-SA. In this case, the binding was sufficiently weak such that all parameters were able to be fit with good confidence (N=0.96±0.08 sites, ΔH=−10.09+1.26 kcal/mol, and K_{D}=6.13±2.16 μM, FIG. 2B). This residual binding is nearly a billion-fold weaker than the natural pair, suggesting that L-biotin will not bind to L-SA in the presence of D-biotin. Without wishing to be bound by theory. these data suggest that BOSS will be highly orthogonal to D-biotin and that BOSS PTI will be suitable for in vivo use without endogenous biotin interference.

### 3. Chemical Synthesis of L-Streptavidin

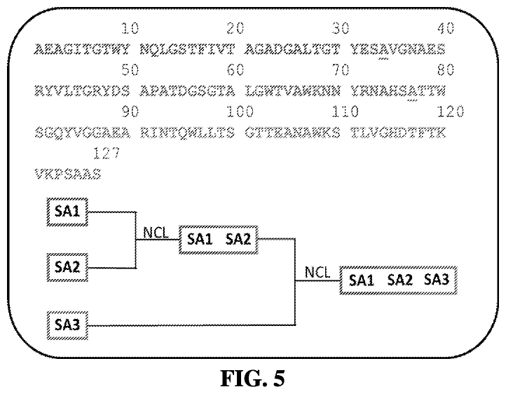

The streptavidin being synthesized is the 127-residue “core” SA,⁴⁶ which has ideal biotin affinity and tetramer stability and is the most commonly used form. L-SA was synthesized prior to the D-synthesis to save on initial troubleshooting cost and to assess the quality of synthesis and folding by comparing synthetic L-SA to recombinant L-SA. This validation was performed because if D-SA is made using the same procedures, it will have identical affinity to L-biotin as synthetic L-SA has to D-biotin. Guided by Aligator²³, a three-segment synthesis strategy (SA1, SA2, and SA3 as shown in FIG. 5). SA1 and SA2 were synthesized with a hydrazide on the C-terminus. SA3 was made as a peptide acid.

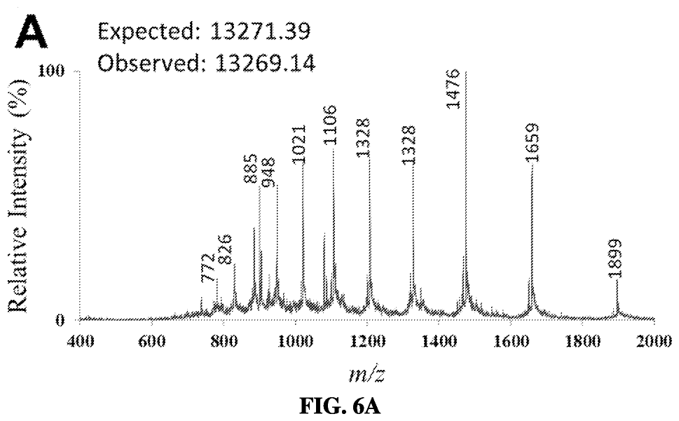

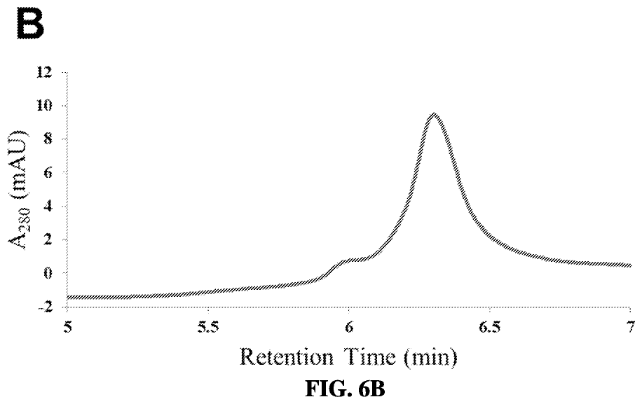

All three peptides were synthesized via SPPS and purified via revese-phase HPLC (RP-HPLC). SAT and SA2 were synthesized with a C-terminal hydrazine for use in NCL after being converted to a thioester. Because SA1 has minimal solubility in ligation buffer, a Helping Hand (HH) molecule was added to the N-terminus. Six Lys residues were then added onto the HH to make K₆-HH-SA1, which has dramatically improved solubility. SA2 and SA3 were soluble enough for NCL without HH modifications. K₆—HH-SA1 was successfully ligated to SA2 to yield K₆-HH-SA1-SA2. Next, SA3 was ligated to K₆-HH-SA1-SA2 to yield K₆-HH-SA1-SA2-SA3. After this final ligation, the HH was cleaved using 1 M hydroxylamine followed by desulfurization to yield SA1-SA2-SA3 (native L-SA) in small quantities. The full-length product was characterized via LC-MS (FIG. 6) and has a small Ala deletion, likely due to an incomplete Ala coupling during the SPPS of SA1.

Peptides were synthesized on a Prelude X instrument (Gyros Protein Technologies) using Fmoc solid-phase peptide synthesis (SPPS) at 50 mol scale. Deprotection cycles employed two treatments of 4 mL 20% piperidine in DMF for 2 min followed by three washes for 30 s using 4 mL DMF. Coupling cycles consisted of addition of 1.3 mL 200 mM amino acid in DMF, 1.3 mL 195 mM HATU in DMF, and 1 mL 600 mM NMM in DMF. Resin and coupling reagents were then mixed using nitrogen bubbling for 25 min at room temperature before being washed three times with 4 mL DMF. In the case of peptide acids (SA3), 0.03 mmol Fmoc-AA was dissolved in 2 mL DMF/DCM, and then 0.3 mmol DIPEA was added to the Fmoc-AA solution. This solution was added to 300 mg of polystyrene 2-cholorotrityl chloride resin and mixed for 1 hour on rotisserie at room temperature. Unreacted groups were capped by rinsing resin with 20 mL of 17:2:1 DCM:MeOH:DIPEA. Polystyrene 2-Chlorotrityl chloride resin was converted to 2-chlorotrityl Fmoc-hydrazine by reacting 300 mg of 2-CTC resin with 61.2 mol of Fmoc-hydrazine in 6 mL of 1:1 DCM and DMF and 0.532 mL DIPEA for 2 hours. Unreacted residues were capped by the addition of 60 L of MeOH and reacting for 10 minutes. Resin was rinsed thoroughly with DCM and DMF. After completion of syntheses, peptide resins were thoroughly washed with DCM and dried under vacuum.

Pseudoproline dipeptides were used to synthesize peptides with sufficient purity. GT, VT, DS, LT, KS, and DT were utilized in the synthesis of SA1-3. TMB-Gly was also used to increase synthesis yield. Underlined Ala residues were synthesized as Cys residues to facilitate native chemical ligation and then desulfurized to yield the native alanine residue.

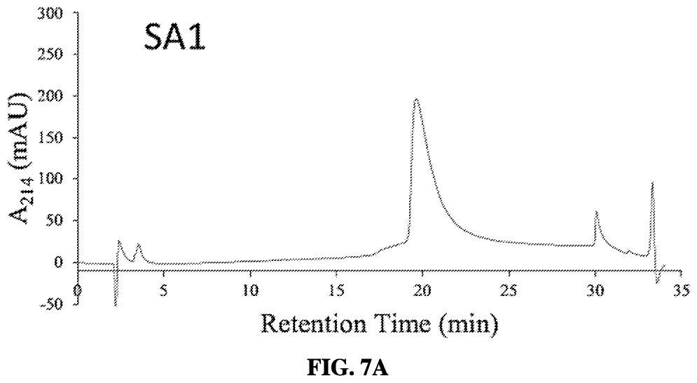

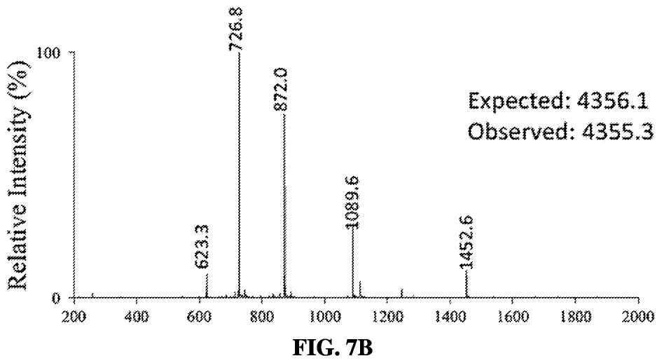

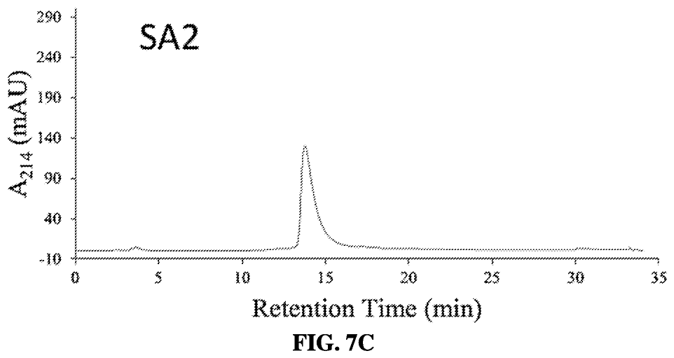

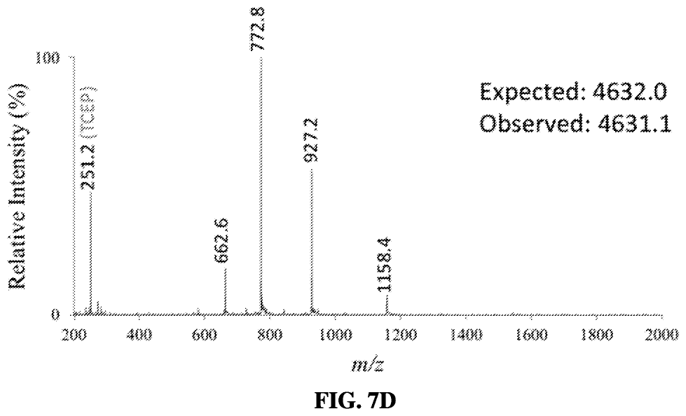

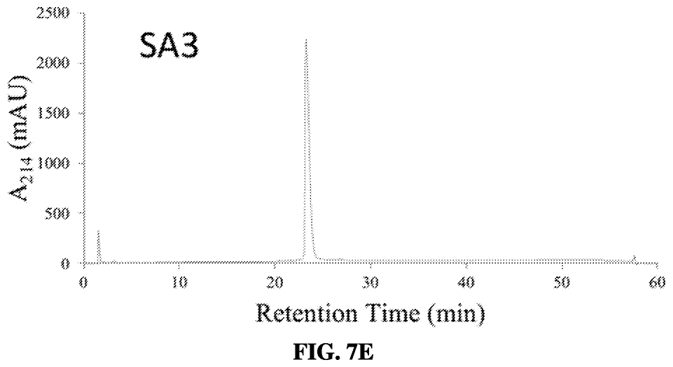

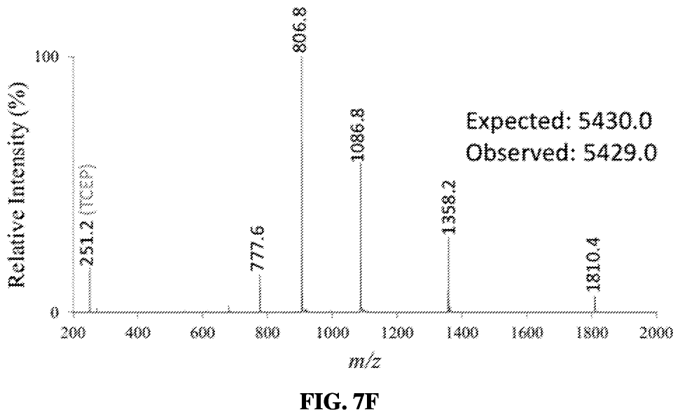

Cleavage of peptide resins was achieved by 3 hours agitation with 6 mL TFA containing 2.5% water and 2.5% TIS per 50 μmol peptide resin. For peptides containing Cys, 2.5% EDT was added to the cleavage cocktail. The TFA solution was precipitated into 40 mL ice-cold diethyl ether per 50 mol crude peptide and centrifuged at 4° C. and 4,000 g for 10 min. The supernatant was decanted while pellets were washed twice with ether before being dissolved in 50% acetonitrile in water and lyophilized overnight. Each peptide was then purified by preparative RP-HPLC with either a C4 (SA1) or C12 (SA2 and SA3) column. The purities of SA1, SA2, and SA3 by RP-HPLC and LC-MS are shown in FIG. 7.

### 4. Native Chemical Ligation, Helping Hand Removal, and Desulfurization

SA1 was not soluble enough for native chemical ligation (NCL), so two separate helping hand strategies were employed. First, a glutamate helping hand was incorporated as an Fmoc-protected amino acid during SPPS at Glu 2 in SA1 and then functionalized with 6 lysine residues. The resulting peptide had greatly increased solubility and underwent NCL with great efficiency, though there were complications with the removal of this helping hand. After this unsuccessful attempt, a Ddap helping hand was added to the N-terminus of SA1 after Fmoc removal of the final residue and the resin was washed with DMF. The resin was agitated in 4 ml 200 mM Ddap in NMP at 37° C. for 24 hours. The resin was washed with DMF, and six lysine residues were added to it via SPPS. This peptide had the same increased solubility and ligation efficiency but Ddap was easily removed.

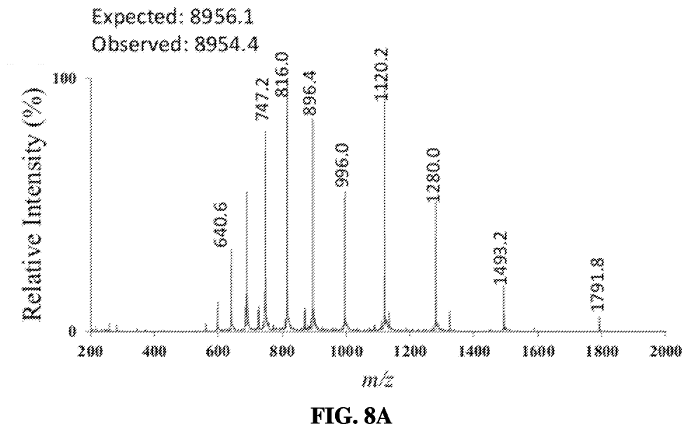

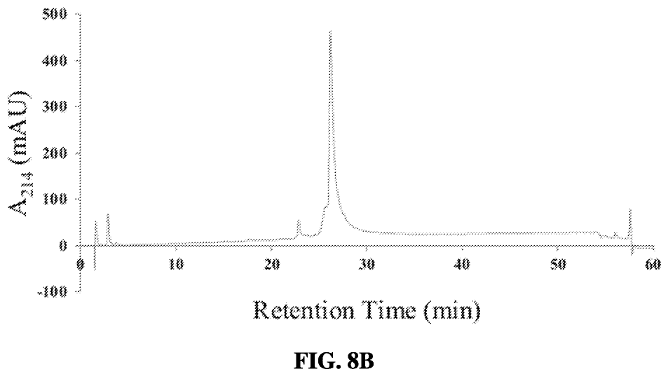

Native chemical ligation reactions were performed according to standard methods using the thiol additive MPAA and TCEP reducing agent. All ligation reactions employed peptide hydrazide method, whereby peptides were dissolved and activated (conversion of hydrazide to acyl azide) in activation buffer (6 M GuHCl, 100 mM phosphate, pH 3) for 20 mm at −20° C. by addition of freshly prepared sodium nitrite solution (15 eq). Following activation, a solution containing freshly prepared MPAA pH 7 in ligation buffer (6 M GuHCl, 100 mM phosphate, pH 7) was added, and the final pH was adjusted to 7 to initiate thiolysis and ligation reaction. Upon completion (based on analytical HPLC and LC/MS), reactions were treated with freshly prepared 150 mM TCEP in 6 M GuHCl, diluted in 5% acetic acid in water, spun at 5000 g, and the supernatant was purified by preparative HPLC. After the second ligation, the helping hand was removed by adding 2 M hydroxylamine in ligation buffer (6 M GuHCl, 100 mM phosphate, pH 7) to a final composition of 1:1 with the ligation reaction volume and reacting at room temperature overnight. The reaction mixture was dialyzed into ligation buffer (6 M GuHCl, 100 mM phosphate, pH 7) over several days, changing the buffer once. Desulfurization was performed using metal-free, radical-mediated procedures. Using desulfurization buffer (6 M GuHCI, 100 mM phosphate, pH 6.5), two stock solutions were prepared: solution A (120 mM VA-044 and 240 mM reduced GSH) and solution B (500 mM TCEP). Equal volumes of solution A and B were added to the dialyzed ligation reaction. The final pH was adjusted to 6.5, and the reactions were stirred at room temperature until desulfurization was complete (˜3 h). The reaction was diluted with 5% AcOH, centrifuged at 5000 g, and the solution was purified by preparative HPLC on a C4 column. The purified ligation product for the SA1 and SA2 ligation is shown in FIG. 8. The desulfurized and purified full-length synthetic streptavidin with the helping hand removed is shown in FIG. 6.

### K. Prophetic Examples

### 1. Optimization of the Chemical Synthesis of L/D-Sa, Characterization, and Pharmacokinetics

### A. Synthesis

Before scaling up the synthesis, troubleshooting the SPPS of SA1 to remove the Ala deletion will be undertaken. The exposed amines will be capped with an acyl group after Ala couplings to prevent peptides that have not completely coupled Ala from moving forward in synthesis. Such capped products are truncated and easily separated during RP-HPLC purification. The synthesis of full-length L-SA can then be scaled-up using previously synthesized SA2 and SA3 via ligation procedures from the small-scale synthesis.

Since NLC is performed under denaturing conditions, L-SA will have to be folded. SA is an extremely stable protein, but recombinant SA can be completely denatured in 6 M guanidine thiocyanate.” This unfolded SA spontaneously refolds (independent of chaperones) when dialyzed into neutral buffer.⁴⁷ The chemically synthesized SA has the same primary amino acid sequence as recombinant SA. Because of this, it will fold under the same conditions as recombinant SA. Once folded, it will have the same activity as recombinant SA. This folding procedure will be reproduced with recombinant SA before using it on L-SA. The optimized synthesis procedures will be used to make D-SA1, D-SA2, and D-SA3 using D-amino acids. The HH is achiral and will add to the N-terminus of an L- or D-peptide. N-terminal K₆-HH will be added on D-SA1 to increase solubility and facilitate NCL. The optimized NCL, HH removal, desulfurization, and folding procedures will be used to make D-SA (D-proteins fold identically to L-proteins, so the optimized procedure is expected to work to fold both enantiomers).

### B. Characterization

After synthesis and folding, the CD spectra of synthetic D- and L-SA will be compared with that of the recombinant protein to confirm the folded state. SA only binds to biotin with high affinity when it is in a tetrameric state. Because of this, it will also be important to analyze the oligomerization state of the protein with analytical ultracentrifugation (AUC) and size-exclusion chromatography (SEC). SEC can also be used to purify the SA tetramer complex and remove aggregates and/or lower order oligomerization states.

As mentioned above, the K_{D} for recombinant L-SA and L-biotin was measured. Whether the binding affinity of the synthetic proteins are the same as the recombinant will be confirmed using ITC. The ITC experiments that were described above will be repeated with synthetic L-SA and then D-SA. It is expected that the data will be nearly identical to what is observed with L-SA. This data will confirm that the correct protein has been made and has been folded correctly.

The binding of L-biotin to recombinant SA is of particular interest. Since D-biotin evolved to bind to SA with such a high affinity, it is surprising that L-biotin binds at all. To illuminate the mechanism behind the difference in binding between the two enantiomers, L-SA will be crystallized with L-biotin and determine its structure via X-ray crystallography. The natural structure (D-biotin/L-SA) will be superimposed with the mismatched structure (L-biotin/L-SA) to determine how SA distinguishes between the biotin enantiomers with billion-fold specificity. Crystal growth conditions using natural SA and biotin have been developed and high-resolution data has been collected. Set drops to co-crystalize SA with L-biotin using the same conditions and L-biotin at millimolar concentrations have been obtained. Preliminary crystals from these conditions are diffracting at sub 2 Å resolution.

### C. Pharmacokinetics

Of interest is also the biodistribution, half-life, and clearance route of D-SA. There is increasing interest in using fluorescence-based full-body imaging (FFI) for collecting these data because of decreased cost and increased safety compared to radiation studies.^{48,50} In collaboration with the University of Utah's Preclinical Research Resource (PRR), FFI will be used to conduct this study. SKH1 hairless mice (Charles River) have been chosen for this study because hair on the mouse can interfere with fluorescence measurements. Alexa Fluor 647 NHS ester (which fluoresces magenta, ThermoFisher) will be added to an amine-functionalized L-biotin to make a fluorescently labeled L-biotin (Mag-L-biotin). Fluorescently labeled D-biotin will also be made using the same method but with Alexa Fluor 568 NHS ester (which fluoresces red, ThermoFisher) to make Red-D-biotin. Next, a 1:1 mixture of Mag-L-biotin to D-SA monomer in PBS and a 1:1 mixture of Red-D-biotin to L-SA in PBS will be made. 4 nmols of the mixtures in 200 μL of PBS will then be intravenously injected into mice, as done previously.^{?}In vivo fluorescence will be measured at 0, 1, 2, 3, 4, 6, 8, 24, and 48 hours using an IVIS Spectrum In Vivo Imaging System (PerkinElmer) and compared to D-SA to L-SA. With each timepoint, the concentration of fluorophore in each organ will be followed. This will demonstrate how D-SA is removed from circulation (i.e., fluorescence concentration in the liver means that it is removed by the liver). The half-life will be determined by quantifying the total level of fluorescence throughout the mouse at the different timepoints. To ensure statistical significance, timepoints and measurements in this study will be performed with 8 mice (group size is estimated by power analysis, 80% power to detect a 50% difference at p<0.05). An equal quantity of male and female mice will be used to account for any differences due to sex as a biological variable.

Collaborating with the PRR, the immunogenicity of D-SA in SKH1 hairless mice will also be tested and compared to L-SA. Groups of 8 mice will be injected intravenously with 30 g⁵¹ of either L- or D-SA at 0, 10, 20, and 40 days. Blood samples will be collected before injections on day 10, 20, and 40 and analyzed using an indirect ELISA assay as done previously.⁵¹ D-SA will be immobilized to the wells of the plate. This is usually accomplished by nonspecific interactions between the plastic of the well and hydrophobic residues on the protein. Since the interaction is not chiral specific, this should work equally well with D-SA as with an L-protein. Blocking will be achieved using bovine serum albumin and then adding a goat anti-mouse IgG secondary antibody-horseradish peroxidase conjugate (ThermoFisher) to see whether any antibody has bound to D-SA. Without wishing to be bound by theory, it is expected that D-SA will have minimal immunogenicity compared to L-SA.

### 2. Demonstration of Boss PTI

### A. Boss Targeting Construct

For a proof-of-concept, a SA PTI method previously described by Cheung et al.⁷ will be replicated using BOSS (BOSS PTI). The single-chain variable fragment (scFv) used in this study that is derived from the 5F11 antibody will be used. An scFv is a fusion protein of the variable regions of the heavy and light chains of an antibody. These small antibody derivatives can be cleared from circulation in <24 hours,^{52,53} much faster than full sized antibodies.^{9,10,20,35} This property is essential for PTI so that the unbound pretargeting agent clears circulation rapidly before the drug is administered. The 5F111 scFv binds to GD₂, which is a glycosphingolipid that is concentrated in gray matter and at synaptic junctions. GD₂ is upregulated in several types of tumors, including neuroblastomas (NB).^{7, 54, 55} It is this upregulation that caused the NIH to rank GD₂ as one of the most promising tumor antigens.⁵⁶ Cheung et al.⁷ expressed their scFv fused to SA. They then used this fusion as their pretargeting tool. Their scFv-SA conjugate was composed of all L-amino acids and recombinantly expressed in F coli. Since there currently is no method to recombinantly express D-proteins, conjugation chemistry will be used to attach chemically synthesized D-SA to the recombinantly expressed L-5F11 scFv. The scFv will be expressed following the published procedure, but a C-terminal sortase tag (Leu-Pro-Xxx-Thr-Gly-Xxx, where Xxx is any amino acid) will be added.⁷ Sortase recognizes this tag and cleaves between Thr and Gly to form an enzyme-bound thioester on the C-terminus of the scFv. Once bound to the scFv, sortase preferentially reacts with the N-terminal amine of an oligo-glycine motif.⁵⁷ Glycine has no chirality, so sortase would recognize an oligo glycine motif appended to the N-terminus of D-SA. This conjugate (scFv-D-SA) will be used for pretargeting experiments. The L-version of this construct (scFv-L-SA) will also be made by recombinant expression, as a control.

### B. Boss PTI in Nb Cells

The efficacy of scFv-D-SA in cells will first be demonstrated using fluorescence microscopy. Neuroblastoma (NB) cells (SK-N-SH from ATCC) and immortalized human cerebral cortex brain cells (HBEC-5i from ATCC) will be cultured. Healthy cerebral cortex (CC) cells have a density of gangliosides that is 5 times lower than NB cells^{55,58-61} and will serve as a control cell line. Four separate experiments will be performed: NB or CC cells with scFv-D-SA and NB or CC cells with scFv-L-SA. A previous study suggests that 5 μM solutions of the scFv constructs with a 3 h incubation period should be sufficient for it to bind to the cells.⁴⁸ After 3 h, the media will be changed and rinsed with PBS buffer to ensure that excess (unbound) scFv-L/D-SA is removed. A 1:1 mixture of Red-D-biotin and Mag-L-biotin (5 M of each) will then be added. After allowing time to bind (3 h), media will be changed and rinsed with buffer. After these steps, the scFv-D-SA and scFv-L-SA should be bound to the NB cells at a much higher concentration than the CC cells, and the biotin conjugates should be bound to D- and L-SA. Whether there is any cross binding of Red-D-biotin and Mag-L-biotin will be determined using fluorescence microscopy. Red-D-biotin should bind exclusively to scFv-L-SA and Mag-L-biotin should bind exclusively to scFv-D-SA. Additionally, a higher fluorescence signal for scFv-D-SA is expected than for scFv-L-SA due to a lack of competing biotin.

### C. Boss PTI in Mice

The procedures described above will be used to determine the half-life and biodistribution of scFv-L-SA and scFv-D-SA in mice using FFI. This study will determine how long it will take for unbound antibody to clear from circulation for the BOSS PTI experiments. Then, a collaboration will be undertaken with the PRR to xenographt NB cells into BALB/c nude mice (Charles River). These mice are immunodeficient so that they will accept the xenograft and are hairless to facilitate FF1. 4 nmols of scFv-D-SA and scFv-L-SA will be administered to xenographed and non-xenographed mice following the previously published procedure.⁷ Next, the empirically determined amount of time for unbound scFv-D-SA and scFv-L-SA to clear will be allowed to pass. 5 nmols⁴⁸ of a 1:1 mixture of Red-D-biotin and Mag-L-biotin will then be injected. The fluorescence will be measured at 0, 1, 2, 3, 4, 6, 8, 24 and 48 hours. It is expected that the fluorescence will localize around the tumor in the xenographed mice and disappear rapidly in the non-xenographed mice. There should be no cross-binding of Red-D-biotin and Mag-L-biotin with BOSS PTI or SA PTI. Additionally, it is expected that the fluorescence signal will be brighter with BOSS PTI than with SA PTI, also due to a lack of interfering biotin.

### L. REFERENCES

1. Howarth, M., Chinnapen, D. J., Gerrow, K., Dorrestein, P. C., Grandy, M. R., Kelleher, N. L., El-Husseini, A., and Ting, A. Y. (2006) A monovalent streptavidin with a single femtomolar biotin binding site, Nat. Methods 3, 267-273.
2. Hohsaka, T., Kajihara, D., Ashizuka, Y., Murakami, H., and Sisido, M. (1999) Efficient incorporation of nonnatural amino acids with large aromatic groups into streptavidin in in vitro protein synthesizing systems, J. Am. Chem. Soc. 121, 34-40.
3. Steen, E. J. L., Edem, P. E., Norregaard, K., Jorgensen, J. T., Shalgunov, V., Kjaer, A., and Herth, M. M. (2018) Pretargeting in nuclear imaging and radionuclide therapy: Improving efficacy of theranostics and nanomedicines, Biomaterials 179, 209-245.
4. Qin, W., Cho, K. F., Cavanagh, P. E., and Ting, A. Y. (2021) Deciphering molecular interactions by proximity labeling, Nat. Methods 18, 133-143.
5. Branon, T. C., Bosch, J. A., Sanchez, A. D., Udeshi, N. D., Svinkina, T., Carr, S. A., Feldman, J. L., Perrimon, N., and Ting, A. Y. (2018) Efficient proximity labeling in living cells and organisms with TurboID, Nat. Biotechnol. 36, 880-887.
6. Schetters, H. (1999) Avidin and streptavidin in clinical diagnostics, Biomol. Eng. 16, 73-78.
7. Cheung, N.-K. V., Modak, S., Lin, Y., Guo, H., Zanzonico, P., Chung, J., Zuo, Y., Sanderson, J., Wilbert, S., and Theodore, L. J. (2004) Single-chain Fv-streptavidin substantially improved therapeutic index in multistep targeting directed at disialoganglioside GD2, J. Nucl. Med. 45, 867-877.
8. Schultz, J., Lin, Y., Sanderson, J., Zuo, Y., Stone, D., Mallett, R., Wilbert, S., and Axworthy, D. (2000) A tetravalent single-chain antibody-streptavidin fusion protein for pretargeted lymphoma therapy, Cancer Res. 60, 6663-6669.
9. Axworthy, D., Reno, J., Hylarides, M., Mallett, R., Theodore, L., Gustavson, L., Su, F.-M., Hobson, L., Beaumier, P., and Fritzberg, A. (2000) Cure of human carcinoma xenografts by a single dose of pretargeted yttrium-90 with negligible toxicity, Proc. Natl. Acad. Sci. 97, 1802-1807.
10. Gu, W., Yudistiro, R., Hanaoka, H., Katsumata, N., and Tsushima, Y. (2021) Potential of three-step pretargeting radioimmunotherapy using biotinylated bevacizumab and succinylated streptavidin in triple-negative breast cancer xenograft, Ann. Nucl. Med. 35, 514-522.
11. Hamblett, K. J., Kegley, B. B., Hamlin, D. K., Chyan, M.-K., Hyre, D. E., Press, O. W., Wilbur, D. S., and Stayton, P. S. (2002) A streptavidin-biotin binding system that minimizes blocking by endogenous biotin, Bioconj. Chem. 13, 588-598.
12. Hamblett, K. J., Press, O. W., Meyer, D. L., Hamlin, D. K., Axworthy, D., Wilbur, D. S., and Stayton, P. S. (2005) Role of biotin-binding affinity in streptavidin-based pretargeted radioimmunotherapy of lymphoma, Bioconj. Chem. 16, 131-138.
13. Parker, C. L., Yang, Q., Yang, B., McCallen, J. D., Park, S. I., and Lai, S. K. (2017) Multivalent interactions between streptavidin-based pretargeting fusion proteins and cell receptors impede efficient internalization of biotinylated nanoparticles, Acta Biomater. 63, 181-189.
14. Press, O. W., Corcoran, M., Subbiah, K., Hamlin, D. K., Wilbur, D. S., Johnson, T., Theodore, L., Yau, E., Mallett, R., and Meyer, D. L. (2001) A comparative evaluation of conventional and pretargeted radioimmunotherapy of CD20-expressing lymphoma xenografts, Blood 98, 2535-2543.
15. Zhang, M., Zhang, Z., Garmestani, K., Schultz, J., Axworthy, D. B., Goldman, C. K., Brechbiel, M. W., Carrasquillo, J. A., and Waldmann, T. A. (2003) Pretarget radiotherapy with an anti-CD25 antibody-streptavidin fusion protein was effective in therapy of leukemia/lymphoma xenografts, Proc. Natl. Acad. Sci. 100, 1891-1895.
16. Park, S. I., Shenoi, J., Frayo, S. M., Hamlin, D. K., Lin, Y., Wilbur, D. S., Stayton, P. S., Orgun, N., Hylarides, M., and Buchegger, F. (2011) Pretargeted radioimmunotherapy using genetically engineered antibody-streptavidin fusion proteins for treatment of non-hodgkin lymphoma, Clin. Cancer Res. 17, 7373-7382.
17. Forster, G. J., Santos, E. B., Smith-Jones, P. M., Zanzonico, P., and Larson, S. M. (2006) Pretargeted radioimmunotherapy with a single-chain antibody/streptavidin construct and radiolabeled DOTA-biotin: strategies for reduction of the renal dose, J. Nucl. Med. 47, 140-149.
18. Yao, Z., Zhang, M., Axworthy, D. B., Wong, K. J., Garmestani, K., Park, L., Park, C. W., Mallett, R. W., Theodore, L. J., and Yau, E. K. (2002) Radioimmunotherapy of A431 xenografted mice with pretargeted B3 antibody-streptavidin and 90Y-labeled 1, 4, 7, 10-tetraazacyclododecane-N, N′, N″, N″′-tetraacetic acid (DOTA)-biotin, Cancer Res. 62, 5755-5760.
19. Zhou, H., Fu, J., Fu, Q., Feng, Y., Hong, R., Li, P., Wang, Z., Huang, X., and Li, F. (2021) Biotin-streptavidin-guided two-step pretargeting approach using PLGA for molecular ultrasound imaging and chemotherapy for ovarian cancer, PeerJ 9, el 1486.
20. Rondon, A., and Degoul, F. (2019) Antibody pretargeting based on bioorthogonal click chemistry for cancer imaging and targeted radionuclide therapy, Bioconj. Chem. 31, 159-173.
21. Del Milton, R., Milton, S., and Kent, S. (1992) Total chemical synthesis of a D-enzyme: The enantiomers of HIV-1 protease show demonstration of reciprocal chiral substrate specificity, Science 256, 1445-1448.
22. Dintzis, H. M., Symer, D. E., Dintzis, R. Z., Zawadzke, L. E., and Berg, J. M. (1993) A comparison of the immunogenicity of a pair of enantiomeric proteins, Proteins 16, 306-308.
23. Jacobsen, M. T., Erickson, P. W., and Kay, M. S. (2017) Aligator: A computational tool for optimizing total chemical synthesis of large proteins, Bioorg. Med. Chem. 25, 4946-4952.
24. Pahler, A., Hendrickson, W. A., Kolks, M., Argarana, C., and Cantor, C. R. (1987) Characterization and crystallization of core streptavidin, J. Biol. Chem. 262, 13933-13937.
25. Weber, P. C., Ohlendorf, D. H., Wendoloski, J., and Salemme, F. (1989) Structural origins of high-affinity biotin binding to streptavidin, Science 243, 85-88.
26. Hyre, D. E., Le Trong, I., Merritt, E. A., Eccleston, J. F., Green, N. M., Stenkamp, R. E., and Stayton, P. S. (2006) Cooperative hydrogen bond interactions in the streptavidin-biotin system, Protein Sci. 15, 459-467.
27. Klumb, L. A., Chu, V., and Stayton, P. S. (1998) Energetic roles of hydrogen bonds at the ureido oxygen binding pocket in the streptavidin-biotin complex, Biochemistry 37, 7657-7663.
28. Schmidt, T. G., and Skerra, A. (1994) One-step affinity purification of bacterially produced proteins by means of the “Strep tag” and immobilized recombinant core streptavidin, J. Chromatogr. A 676, 337-345.
29. Thie, H., Voedisch, B., D0bel, S., Hust, M., and Schirrmann, T. (2009) Affinity maturation by phage display, In Therapeutic Antibodies, pp 309-322, Springer.
30. Piketty, M.-L., Polak, M., Flechtner, I., Gonzales-Briceno, L., and Souberbielle, J.-C. (2017) False biochemical diagnosis of hyperthyroidism in streptavidin-biotin-based immunoassays: the problem of biotin intake and related interferences, Clin. Chem. Lab. 55, 780-788.
31. Ardabilygazir, A., Afshariyamchlou, S., Mir, D., and Sachmechi, I. (2018) Effect of high-dose biotin on thyroid function tests: case report and literature review, Cureus 10.
32. Li, J., Wagar, E. A., and Meng, Q. H. (2018) Comprehensive assessment of biotin interference in immunoassays, Clin. Chim. Acta 487, 293-298.
33. Frame, 1. J., Joshi, P. H., Mwangi, C., Gunsolus, I., De Lemos, J. A., Das, S. R., Sarode, R., Balani, J., Apple, F. S., and Muthukumar, A. (2019) Susceptibility of cardiac troponin assays to biotin interference, Am. J. Clin. Pathol. 151, 486-493.
34. Liu, G. (2018) A revisit to the pretargeting concept-A target conversion, Front. Pharmacol. 9, 1476.
35. Sarrett, S. M., Keinanen, O., Dayts, E. J., Dewaele-Le Roi, G., Rodriguez, C., Carnazza, K. E., and Zeglis, B. M. (2021) Inverse electron demand Diels-Alder click chemistry for pretargeted PET imaging and radioimmunotherapy, Nat. Protoc., 1-34.
36. Knight, J. C., and Comelissen, B. (2014) Bioorthogonal chemistry: implications for pretargeted nuclear (PET/SPECT) imaging and therapy, Am. J. Nucl. Med. Mol. Imaging 4, 96.
37. Jallinoja, V. I., and Houghton, J. L. (2021) Current Landscape in Clinical Pretargeted Radioimmunoimaging and Therapy, J. Nucl. Med. 62, 1200-1206.
38. Srisa-Art, M., Dyson, E. C., deMello, A. J., and Edel, J. B. (2008) Monitoring of real-time streptavidin-biotin binding kinetics using droplet microfluidics, Anal. Chem. 80, 7063-7067.
39. Chilkoti, A., and Stayton, P. S. (1995) Molecular origins of the slow streptavidin-biotin dissociation kinetics, J. Am. Chem. Soc. 117, 10622-10628.
40. Suganuma, M., Kubo, T., Ishiki, K., Tanaka, K., Suto, K., Ejima, D., Toyota, M., Tsumoto, K., Sato, T., and Nishikawa, Y. (2022) Mirror-image Streptavidin with Specific Binding to “Non-natural” L-biotin, Pre-Print, Research Square.
41. Behrendt, R., White, P., and Offer, J. (2016) Advances in Fmoc solid-phase peptide synthesis, J. Pept. Sci. 22, 4-27.
42. Dawson, P. E., Muir, T. W., Clarklewis, I., and Kent, S. B. H. (1994) Synthesis of Proteins by Native Chemical Ligation, Science 266, 776-779.
43. Yan, L. Z., and Dawson, P. E. (2001) Synthesis of peptides and proteins without cysteine residues by native chemical ligation combined with desulfurization, J. Am. Chem. Soc. 123, 526-533.
44. Fulcher, J. M., Petersen, M. E., Giesler, R. J., Cruz, Z. S., Eckert, D. M., Francis, J. N., Kawamoto, E. M., Jacobsen, M. T., and Kay, M. S. (2019) Chemical synthesis of Shiga toxin subunit B using a next-generation traceless “helping hand” solubilizing tag, Org. Biomol. Chem. 17, 10237-10244.
45. Jacobsen, M. T., Petersen, M. E., Ye, X., Galibert, M., Lorimer, G. H., Aucagne, V., and Kay, M. S. (2016) A helping hand to overcome solubility challenges in chemical protein synthesis, J. Am. Chem. Soc. 138, 11775-11782.
46. Sano, T., Pandori, M. W., Chen, X., Smith, C. L., and Cantor, C. R. (1995) Recombinant core streptavidins: a minimum-sized core streptavidin has enhanced structural stability and higher accessibility to biotinylated macromolecules, J. Biol. Chem. 270, 28204-28209.
47. Kurzban, G., Bayer, E., Wilchek, M., and Horowitz, P. (1991) The quaternary structure of streptavidin in urea, J. Biol. Chem. 266, 14470-14477.
48. Zhang, X., Wang, B., Zhao, N., Tian, Z., Dai, Y., Nie, Y., Tian, J., Wang, Z., and Chen, X. (2017) Improved tumor targeting and longer retention time of NIR fluorescent probes using bioorthogonal chemistry, Theranostics 7, 3794.
49. Bojkowska, K., De Sio, F. S., Barde, I., Offner, S., Verp, S., Heinis, C., Johnsson, K., and Trono, D. (2011) Measuring in vivo protein half-life, Chem. Biol. 18, 805-815.
50. Meng, F., Wang, J., Ping, Q., and Yeo, Y. (2018) Quantitative assessment of nanoparticle biodistribution by fluorescence imaging, revisited, ACS Nano 12, 6458-6468.
51. Chinol, M., Casalini, P., Maggiolo, M., Canevari, S., Omodeo, E., Caliceti, P., Veronese, F., Cremonesi, M., Chiolerio, F., and Nardone, E. (1998) Biochemical modifications of avidin improve pharmacokinetics and biodistribution, and reduce immunogenicity, Br. J. Cancer 78, 189-197.
52. Wu, A. M., Chen, W., Raubitschek, A., Williams, L. E., Neumaier, M., Fischer, R., Hu, S.-z., Odom-Maryon, T., Wong, J. Y., and Shively, J. E. (1996) Tumor localization of anti-CEA single-chain Fvs: improved targeting by non-covalent dimers, Immunotechnology 2, 21-36.
53. Asano, R., Koyama, N., Hagiwara, Y., Masakari, Y., Orimo, R., Arai, K., Ogata, H., Furumoto, S., Umetsu, M., and Kumagai, I. (2016) Anti-EGFR scFv tetramer (tetrabody) with a stable monodisperse structure, strong anticancer effect, and a long in vivo half-life, FEBS open bio 6, 594-602.
54. Richman, S. A., Nunez-Cruz, S., Moghimi, B., Li, L. Z., Gershenson, Z. T., Mourelatos, Z., Barrett, D. M., Grupp, S. A., and Milone, M. C. (2018) High-affinity GD2-specific CAR T cells induce fatal encephalitis in a preclinical neuroblastoma model, Cancer Immunol. Res. 6, 36-46.
55. Rodden, F. A., Wiegandt, H.. and Bauer, B. L. (1991) Gangliosides: the relevance of current research to neurosurgery, J. Neurosurg. 74, 606-619.
56. Cheever, M. A., Allison, J. P., Ferris, A. S., Finn, O. J., Hastings, B. M., Hecht, T. T., Mellman, I., Prindiville, S. A., Viner, J. L., and Weiner, L. M. (2009) The prioritization of cancer antigens: a national cancer institute pilot project for the acceleration of translational research, Clin. Cancer Res. 15, 5323-5337.
57. Guimaraes, C. P., Witte, M. D., Theile, C. S., Bozkurt, G., Kundrat, L., Blom, A. E., and Ploegh, H. L. (2013) Site-specific C-terminal and internal loop labeling of proteins using sortase-mediated reactions, Nat. Protoc. 8, 1787-1799.
58. Wu, Z.-L., Schwartz, E., Seeger, R., and Ladisch, S. (1986) Expression of GD2 ganglioside by untreated primary human neuroblastomas, Cancer Res. 46, 440-443.
59. Schengrund, C. L., Repman, M. A., and Shochat, S. J. (1985) Ganglioside composition of human neuroblastomas correlation with prognosis A pediatric oncology group study, Cancer 56, 2640-2646.
60. LAMMIE, G. A., Cheung, N., GERALD, W., ROSENBLUM, M., and CORDONCARDO, C. (1993) Ganglioside gd (2) expression in the human nervous-system and in neuroblastomas-an immunohistochemical study, Int. J. Oncol. 3, 909-915.
61. Eto, Y., and Shinoda, S. (1982) Gangliosides and neutral glycosphingolipids in human brain tumors: specificity and their significance, Adv. Exp. Med. Biol. 152, 279-290.
62. Witte, M. D., Theile, C. S., Wu, T., Guimaraes, C. P., Blom, A. E., and Ploegh, H. L. (2013) Production of unnaturally linked chimeric proteins using a combination of sortase-catalyzed transpeptidation and click chemistry, Nat. Protoc. 8, 1808-1819.
63. Cho, H., Daniel, T., Buechler, Y. J., Litzinger, D. C., Maio, Z., Putnam, A. M., Kraynov, V. S., Sim, B. C., Bussell, S., Javahishvili, T., Kaphle, S., Viramontes, G., Ong, M., Chu, S., Becky, G. C., Lieu, R., Knudsen, N., Castiglioni, P., Norman, T. C., Axelrod, D. W., Hoffman, A. R., Schultz, P. G., DiMarchi, R. D., and Kimmel, B. E. (2011) Optimized clinical performance of growth hormone with an expanded genetic code, Proc. Natl. Acad. Sci. USA 108, 9060-9065.
64. Mu, J., Pinkstaff, J., Li, Z., Skidmore, L., Li, N., Myler, H., Dallas-Yang, Q., Putnam, A.-M., Yao, J., and Bussell, S. (2012) FGF21 analogs of sustained action enabled by orthogonal biosynthesis demonstrate enhanced antidiabetic pharmacology in rodents, Diabetes 61, 505-512.
65. Ou, W. J., Uno, T., Chiu, H. P., Grunewald, J., Cellitti, S. E., Crossgrove, T., Hao, X. S., Fan, Q., Quinn, L. L., Patterson, P., Okach, L., Jones, D. H., Lesley, S. A., Brock, A., and Geierstanger, B. H. (2011) Site-specific protein modifications through pyrroline-carboxy-lysine residues, Proc. Natl. Acad. Sci. 108, 10437-10442.
66. Dumas, A., Spicer, C. D., Gao, Z., Takehana, T., Lin, Y. A., Yasukohchi, T., and Davis, B. G. (2013) Self-liganded Suzuki-Miyaura coupling for site-selective protein PEGylation, Angew. Chem. Int. Ed. 52, 3916-3921.
67. Wilding, K. M., Smith, A. K., Wilkerson, J. W., Bush, D. B., Knotts IV, T. A., and Bundy, B. C. (2018) The locational impact of site-specific PEGylation: streamlined screening with cell-free protein expression and coarse-grain simulation, ACS Synth. Biol. 7, 510-521.
68. Junghans, R., and Anderson, C. (1996) The protection receptor for IgG catabolism is the beta2-microglobulin-containing neonatal intestinal transport receptor, Proc. Natl. Acad. Sci. 93, 5512-5516.
69. Vieira, P., and Rajewsky, K. (1988) The half-lives of serum immunoglobulins in adult mice, Eur. J. Immunol. 18, 313-316.
70. Chivers, C. E., Crozat, E., Chu, C., Moy, V. T., Sherratt, D. J., and Howarth, M. (2010) A streptavidin variant with slower biotin dissociation and increased mechanostability, Nat. Methods 7, 391-393.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A bi-specific polypeptide comprising: an antibody or antibody fragment thereof covalently linked to D-streptavidin (D-SA) or a variant thereof.

2. The bi-specific polypeptide of claim 1, wherein the antibody or antibody fragment thereof is an anti-IL-17 receptor antibody, an anti-IL-5 receptor antibody, an anti-PD-L1 antibody, an anti-FGF23 antibody, an anti-epithelial growth factor receptor antibody, an anti-GD2 antibody, an anti-HER-2 receptor antibody, an anti-RANKL antibody, an anti-C5 antibody, an anti-VEGF receptor antibody, an anti-VEGF-A antibody, an anti-VEGF receptor-2 antibody, anti-IgE antibody, an anti-TNF-alpha antibody, an anti-IL-12/23 antibody, an anti-CTLA-4 antibody, an anti-CD30 antibody, an anti-CD4 antibody, an anti-CGRP receptor antibody, an anti-CD3 antibody, an anti-CD20 antibody, an anti-CD25 antibody, or an anti-GP11b/11a antibody.

3. The bi-specific polypeptide of claim 1, wherein the antibody or antibody fragment thereof is an anti-CD30 antibody.

4. The bi-specific polypeptide of claim 1, wherein the antibody or antibody fragment thereof specifically binds a cell surface marker.

5. The bi-specific polypeptide of claim 4, wherein the cell surface marker is CD3, CD4, CD5, CD20, CD25, or a glycosphingolipid.

6. The bi-specific polypeptide of claim 5, wherein the glycosphingolipid is GD₂.

7. The bi-specific polypeptide of claim 1, wherein the antibody or antibody fragment thereof is a single chain antibody (scFv) or a F′ab fragment.

8. The bi-specific polypeptide of claim 7, wherein the scFv is derived from burosumab, ibalizumab, erenumab, atezolizumab, reslizumab, pembrolizumab, nivolumab, ramucirumab, ipilimumab, brentuximab, ustekinumab, panitumaumab, ranibizumab, necitumaumab, dinutuximab, denosumab, exulizumab, bevacizumab, omalizumab, adalimumab, avelumab, durvalumab, brodalumab, muromonoab-CD3, abciximab, rituximab, daclizumab, infliximab, basiliximab, palivizumab, trastuzumab, gemtuzumab or a biologically active variant thereof.

9. The bi-specific polypeptide of claim 1, wherein the antibody or antibody fragment thereof is covalently linked to D-SA.

10. The bi-specific polypeptide of claim 1, wherein the antibody or antibody fragment thereof is covalently linked to a variant of D-SA.

11. The polypeptide of claim 10, wherein the variant of D-SA is D-traptavidin, D-strep-tactin, D-strep-tactin XT, or monovalent D-SA.

12. The bi-specific polypeptide of claim 1, wherein the D-SA or variant thereof specifically binds L-biotin.

13-15. (canceled)

16. A pharmaceutical composition comprising an effective amount of the bi-specific polypeptide of claim 1 and a pharmaceutically acceptable carrier.

17. The pharmaceutical composition of claim 16, wherein the pharmaceutical composition is formulated for intravenous administration.

18. A method of treating a disease or a condition in a subject in need thereof, the method comprising:
(a) administering to the subject an effective amount of the bi-specific polypeptide of claim 1; and
(b) subsequently administering to the subject an effective amount of a composition comprising L-biotin covalently linked to a therapeutic agent,
wherein the D-SA or the variant thereof specifically binds L-biotin.

19-24. (canceled)

25. The method of claim 18, wherein the disease is cancer, non-Hodgkin lymphoma, multiple sclerosis, Crohn's disease, rheumatoid arthritis, asthma, macular degeneration, psoriasis, Hodgkin lymphoma, paroxysmal nocturnal hemoglobinuria, or X-linked hypophosphatemia.

26-29. (canceled)

30. The method of claim 18, wherein the condition is prevention of blood clots in angioplasty, kidney transplantation rejection, migraine prevention, HIV infection, or bone loss.

31. (canceled)

32. The method of claim 18, wherein the therapeutic agent is a radioactive agent or a chemotherapeutic agent.

33-47. (canceled)

48. A conjugate comprising L-biotin covalently linked to a therapeutic agent or a diagnostic agent.

49. The conjugate of claim 48, wherein the therapeutic agent is a chemotherapeutic agent or a cancer diagnostic agent.

50-91. (canceled)


SEQUENCE LISTING (11 sequences)

SEQ ID NO: 1 - protein, 33 aa, synthetic construct
  VARIANT 1..33: note = Each of amino acids 1-33 can be a D- or L- amino acid
AEAGITGTWYNQLGSTFIVTAGADGALTGTYES

SEQ ID NO: 2 - protein, 33 aa, synthetic construct; identical to SEQ ID NO: 1
  VARIANT 1..33: note = Each of amino acids 1-33 can be a D- or L- amino acid
  SITE 33: note = Hydrazine formed amino acid

SEQ ID NO: 3 - protein, 43 aa, synthetic construct
  VARIANT 1..43: note = Each of amino acids 1-43 can be a D- or L- amino acid
AVGNAESRYVLTGRYDSAPATDGSGTALGWTVAWKNNYRNAHS

SEQ ID NO: 4 - protein, 43 aa, synthetic construct; identical to SEQ ID NO: 3
  VARIANT 1..43: note = Each of amino acids 1-43 can be a D- or L- amino acid
  SITE 43: note = Hydrazine formed amino acid

SEQ ID NO: 5 - protein, 51 aa, synthetic construct
  VARIANT 1..51: note = Each of amino acids 1-51 can be a D- or L- amino acid
ATTWSGQYVGGAEARINTQWLLTSGTTEANAWKSTLVGHDTFTKVKPSAAS

SEQ ID NO: 6 - protein, 33 aa, synthetic construct; identical to SEQ ID NO: 1
  VARIANT 1..33: note = Each of amino acids 1-33 can be a D- or L- amino acid
  SITE 33: note = Hydrazine formed amino acid
  SITE 1: note = The N-terminal end of the amino acid is conjugated with a linker

SEQ ID NO: 7 - protein, 33 aa, synthetic construct; identical to SEQ ID NO: 1
  VARIANT 1..33: note = Each of amino acids 1-33 can be a D- or L- amino acid
  SITE 33: note = Hydrazine formed amino acid
  SITE 1: note = The N-terminal end of the amino acid is conjugated with a linker

SEQ ID NO: 8 - protein, 127 aa, synthetic construct
  VARIANT 1..127: note = Each of amino acids 1-127 can be a D- or L- amino acid
AEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 9 - protein, 76 aa, synthetic construct
  VARIANT 1..76: note = Each of amino acids 1-76 can be a D- or L- amino acid
  SITE 76: note = Hydrazine formed amino acid
  SITE 1: note = The N-terminal end of the amino acid is conjugated with a linker
AEAGITGTWYNQLGSTFIVTAGADGALTGTYESAVGNAESRYVLTGRYDSAPATDGSGTALGWTVAWKNNYRNAHS

SEQ ID NO: 10 - protein, 127 aa, synthetic construct; identical to SEQ ID NO: 8
  VARIANT 1..127: note = Each of amino acids 1-127 can be a D- or L- amino acid
  SITE 1: note = The N-terminal end of the amino acid is conjugated with a linker

SEQ ID NO: 11 - protein, 6 aa, synthetic construct
  VARIANT 1..6: note = Each of amino acids 1-6 can be a D- or L- amino acid
  SITE 6: note = The C-terminal end of the amino acid is conjugated with a linker
KKKKKK